(19) United States
(12) Patent Application Publication
(10) Pub. No.: US 2022/0301718 A1
(43) Pub. Date: Sep. 22, 2022

(54) System, Device, and Method of Determining Anisomelia or Leg Length Discrepancy (LLD) of a Subject by Using Image Analysis and Machine Learning

(71) Applicants: Seshadri Paravastu, San Jose, CA (US); Radha Samavedam Paravastu, San Jose, CA (US); Keshavan Hemmige Srivatsan, San Jose, CA (US)

(72) Inventors: Seshadri Paravastu, San Jose, CA (US); Radha Samavedam Paravastu, San Jose, CA (US); Keshavan Hemmige Srivatsan, San Jose, CA (US)

(21) Appl. No.: 17/717,072

(22) Filed: Apr. 9, 2022

Related U.S. Application Data
(63) Continuation-in-part of application No. 17/203,499, filed on Mar. 16, 2021, now Pat. No. 11,322,244.

(51) Int. Cl. G16H 50/20; G16H 30/20

(52) U.S. Cl. CPC G16H 50/20; G16H 30/20

(57) ABSTRACT

System, device, and method of determining Anisomelia or Leg Length Discrepancy (LLD) of a subject, by using image analysis and machine learning. A system includes a plurality of end-user devices; each device includes a camera to capture digital non-radiological non-X-Ray photographs of legs of a person; each device further includes a local Deep Neural Network (DNN) engine to perform local classification of images as either manifesting LLD or non-manifesting LLD. The digital non-radiological non-X-Ray photographs are also uploaded from the end-user devices to a central server, which updates and upgrades the DNN model based on transfer learning, and periodically distributes the upgraded DNN model downstream to the end-user devices.

### CROSS-REFERENCE TO RELATED APPLICATIONS

This patent application is a Continuation-in-Part (CIP) of U.S. Ser. No. 17/203,499, filed on Mar. 16, 2021, which is hereby incorporated by reference in its entirety.

### FIELD

Some embodiments are related to the field of computerized systems for determining medical conditions.

### BACKGROUND

Anisomelia, or Leg Length Discrepancy (LLD) or Leg Length Inequality (LLI), is a condition in which the two legs of a human have unequal length. Some researchers estimate that such condition may affect between 40 to 70 percent of the general population.

Some cases of LLD may be caused by structural or anatomical differences between the two legs; for example, due to differences in the length of the femur in the thigh, or the tibia bone or fibula bone in the lower leg. Some cases of LLD may be caused by a birth defect, or due to a broken leg or a severe infection.

### SUMMARY

Some embodiments provide systems, devices, and methods of determining or detecting Anisomelia or Leg Length Discrepancy (LLD) of a human subject, by using image analysis and machine learning. A system includes a plurality of end-user devices; each device includes a camera to capture digital non-radiological non-X-Ray photographs of legs of a person. Each device further includes a local Deep Neural Network (DNN) engine to perform local classification of images as either manifesting LLD or non-manifesting LLD. The digital non-radiological non-X-Ray photographs are also uploaded from the end-user devices to a central server, which updates and upgrades the DNN model based on transfer learning, and periodically distributes the upgraded DNN model downstream to the end-user devices.

Some embodiments may provide other and/or additional benefits or advantages.

### BRIEF DESCRIPTION OF THE DRAWINGS

FIG. 1 is a schematic illustration of a system, including the LEG-Minder client-side device and the LEGislator server-side device, the system being operable for Leg Length Discrepancy (LLD) detection or diagnosis, in accordance with some demonstrative embodiments.

FIG. 2 is a block diagram illustration of System Level Architecture of the LEG-Minder device and the LEGislator device combination, in accordance with some demonstrative embodiments.

FIG. 3 is a block diagram illustration of Component Level architecture of the LEG-Minder device which forms the patient image portion, in accordance with some demonstrative embodiments.

FIG. 4 is a block diagram illustration of the LEGislator Master Neural Network Server and the Learning server which forms the cloud-based algorithm updater portion in accordance with some demonstrative embodiments.

FIG. 5 is an illustration demonstrating Step A, which is “Initial Learning” by the LEGislator server, in accordance with some demonstrative embodiments.

FIG. 6 is an illustration demonstrating Step B, which is “Upgrade LEG Minder”, or the process of upgrading the LEG-Minder by the LEGislator, in accordance with some demonstrative embodiments.

FIG. 7 is an illustration demonstrating Step C, which is “Transfer Data”, which is the steps that are involved with LEG-Minder transfer of data, in accordance with some demonstrative embodiments.

FIG. 8 is an illustration demonstrating Step D, which is “Transfer Learning”, which includes the steps involved with transfer learning by LEGislator, in accordance with some demonstrative embodiments.

FIG. 9 is an illustration demonstrating Step E, which is “Mission Mode”, which includes the steps involved with operational mode of LEG-Minder, in accordance with some demonstrative embodiments.

FIG. 10 is an illustration demonstrating Pipeline Architecture for Neural Network Model Generation, and further demonstrating the Learning server's pipeline architecture, in accordance with some demonstrative embodiments.

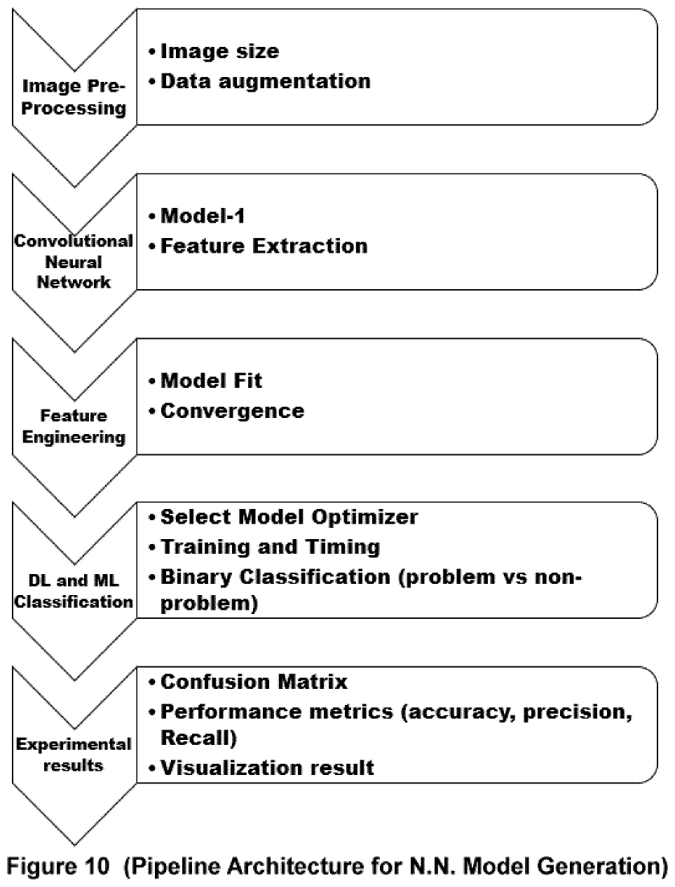

FIG. 11 is a flow-chart demonstrating a user flow with the LEG-Minder device, in accordance with some demonstrative embodiments.

FIG. 12 is an illustration demonstrating Convolution and pooling, which shows the configurations and the summary of transformations, in accordance with some demonstrative embodiments.

FIG. 13 is an illustration demonstrating Convolutional layers and Max Pooling, which shows the traversal of an image though all the convolutional and the pooling layers, in accordance with some demonstrative embodiments.

FIG. 14 is a flow-chart demonstrating Image Pre-Processing, in accordance with some demonstrative embodiments.

FIG. 15 is an illustration demonstrating Convolution Building and the architecture of the convolutional neural network, in accordance with some demonstrative embodiments.

FIG. 16 is an illustration demonstrating Padding Implementation and how padding is implemented in the algorithm, in accordance with some demonstrative embodiments.

FIG. 17 is an illustration demonstrating Dimensionality Reduction and how performance improvements may be implemented to achieve dimensionality reduction, in accordance with some demonstrative embodiments.

FIG. 18 is an illustration demonstrating the Deep Neural Network (DNN) and the implementation of back propagation, in accordance with some demonstrative embodiments.

FIG. 19 is a set of two illustrations demonstrating Laplacian Edge Detection for an image of tibia and fibula, in accordance with some demonstrative embodiments.

FIG. 20 is an illustration demonstrating Bone Contour Detection, in accordance with some demonstrative embodiments.

FIG. 21 is an illustration demonstrating Bone Contouring, in accordance with some demonstrative embodiments.

FIG. 22 is an illustration demonstrating Rectangular Contouring (with rotated and non-rotated rectangular boundary), in accordance with some demonstrative embodiments.

FIG. 23 is an illustration demonstrating Minima Detection, in accordance with some demonstrative embodiments.

FIG. 24 is an illustration demonstrating LLD detection, in accordance with some demonstrative embodiments.

FIG. 25 is a flowchart demonstrating point fitting via a Machine Learning (ML) engine, in accordance with some demonstrative embodiments.

FIG. 26 is an illustration demonstrating Sources of Data Retrieval, in accordance with some demonstrative embodiments.

FIG. 27 is a set of illustrations demonstrating various leg posture photographs that can be utilized for LLD detection, in accordance with some demonstrative embodiments.

FIG. 28 is a set of illustrations demonstrating photograph capturing of various leg postures, in accordance with some demonstrative embodiments.

FIG. 29 is a flowchart of processing or utilizing photographs or non-X-Ray images, in accordance with some demonstrative embodiments.

FIG. 30 is an illustration demonstrating optional body landmarks and their locations, which may be used in accordance with some demonstrative embodiments.

### DETAILED DESCRIPTION OF SOME DEMONSTRATIVE EMBODIMENTS

Anisomelia or Leg length discrepancy (LLD) is a condition involving abnormal loading of the lower extremity and lumbar joints, and/or unequal length of the two legs of a human. While simple X-rays may attempt to diagnose some cases of LLD, it is estimated that only one-third of the world's population has X-Ray diagnostic imaging access. There is a need to provide an efficient, user-friendly, cost-effective, portable, solution that may allow end-users (including individual users, or remote medical offices that do not have cumbersome and costly X-Ray equipment and X-Ray personnel) to efficiently diagnose LLD. The proposed solution of some embodiments elegantly and efficiently provides and strengthens the processes for the assessment, adoption, and use of appropriate health technologies for diagnostic imaging with digital photographs (e.g., captured by a smartphone or a cellular phone or a tablet, or a web-camera or web-cam, or a gaming device or other camera-equipped electronic device) and avoids the need to install, maintain and operate expensive for X-ray equipment, and/or can become a substitute for cases that need radiation protection of the public, workers, patients, and/or the environment.

Anatomic leg-length inequality is near-universal. It is estimated that Leg length discrepancy (LLD) affects up to 90% of the general population, with a mean discrepancy of 5.2 millimeters between the length of two legs of a human. In most such cases, LLD is typically mild (e.g., the length discrepancy is smaller than 20 mm). When overlooked during early medical examinations, severe spinal cord misalignment and/or kyphosis may occur in children born with this condition. Therefore, early detection may be vital in Anisomelia. The system of some embodiments includes inexpensive, affordable, reachable, and non-radiology equipment to perform a test to detect LLD which is very much needed, as realized by the Applicants. Hence the need for an innovative system that can detect this condition. Some of the valuable features for new inspection method and system of some embodiments may include: (a) No radiation exposure; no need for costly and bulky X-Ray equipment; (b) Equally accurate to, or more accurate than, current X-Ray based inspection methods; (c) More cost-effective, affordable, cheaper, and smaller form-factor solution.

The present invention is a technology relating to a Leg Length Discrepancy (LLD) diagnosis or detection system, using Machine Learning (ML) and/or Deep Learning (DL) and/or Deep Neural Network (DNN) learning and/or other suitable type of Artificial Intelligence (AI) engine or unit. It comprises a diagnosis “Leg-Minder” device that is installed in (or is available at) each diagnosis center, typically as a client-side device, and determines the patients legs to either have a height (or length) discrepancy (e.g., beyond a pre-defined threshold value), or not, on the basis of a neural network model which utilizes a patient legs photograph or a potential radiographic image (e.g., an X-ray image which can still be used in some implementation, instead of non-X-Ray images or in addition to them) as inputs; and a neural network learning server which is the “LEGislator” that is connected to the Internet (or to a local or area or regional communication network) and performs DNN Learning on the LLD database of a plurality of “LEG-Minder” devices in the network. It is noted that the terms “LEGislator” or “LEG-islator” are used herein as a non-limiting example of a server or a central server or a computer or a computing device or a cloud-computing server, which may perform the functionalities described above and/or herein as the Server side of a demonstrative client-server architecture; and similarly, the terms “LEG-Minder” or “LEGminder” are used herein as a non-limiting example of a client-side computing device or electronic device, which may perform the functionalities described above and/or herein as the Client side of a demonstrative client-server architecture.

For demonstrative purposes, some portions of the discussion above or herein may refer to a smartphone or a tablet as non-limiting examples of electronic devices that may be used, in some embodiments, for capturing or acquiring (and uploading/transferring) digital photographs of legs of human subjects; however, other suitable devices may be used for this purposes; for example, an electronic device that is equipped with a camera or imager and that is capable of transferring-out data or uploading data via a Wi-Fi connection or a wireless connection or a cellular connection or a Bluetooth connection or via a wired link or a cable; a digital camera or a stand-alone camera; a laptop computer equipped with a camera; a desktop computer equipped with a camera; or a special-purpose electronic device that is equipped with a camera, a processor, a memory unit, and a wireless (or wired) transceiver for uploading or transferring captured images.

In particular, some embodiments of the present invention relate to a technology or system in which patient's leg photos and diagnostic result data are acquired or captured or imaged in each diagnosis center, and are then uploaded to a centralized server which is the neural network learning server or “LEGislator device”. Then, on the basis of this collected or aggregated information, the learning server performs DNN learning or training on its neural network model, to generate an initial and then an upgraded neural network model. This dynamically-updated and upgraded model is later downloaded (e.g., periodically, such as once per day or once per week) to all the LEG-Minder devices in the network. Therefore, the LEG-Minder device becomes an integral part of a neural network system and model, which is optimized to the diagnosis environment within a diagnosis center.

Anisomelia is classified as either anatomical (structural) or functional. Structural is side-to-side differences in lower limb length, while functional is due to bio-mechanical abnormalities of joint function in the lower limbs (athletes). The causes for LLD can be congenital or can be acquired. Congenital causes include, for example, phocomelia and dysgenetic syndromes. Acquired causes include, for example: dysplasias, Ollier's disease, polio, osteomyelitis, neurofibromatosis; septic arthritis; fractures; and surgically induced, or due to breaking a leg, or due to severe infection. LLD can exist from childhood, or it can develop in adult life. The clinical methods (direct and indirect methods) in common use to measure leg length discrepancy (LLD) cannot always meet the demands of precision and accuracy, or are not reachable or accessible or affordable at rural locations or non-urban locations. Some of the current clinical methods of assessing this discrepancy include manual tape measures of the leg lengths, and X-rays for measuring bones length. Some researchers estimate that clinical assessments of this condition were incorrect by at least 5 mm in at least 29% of subjects.

In addition, it also turns out that these conventional methods are expensive (both time-wise and cost-wise), and not necessarily prescribed to every patient due to undesired exposure to X-Ray radiation of the pelvic region or other body organs which may also be inadvertently exposed to X-Ray, which may be unsafe for some patients (e.g., young children).

The conventional manual diagnosis can be complicated and error-prone. For example, the attending physician needs to clearly observe the patient's posture and needs to then autonomously suspect LLD condition. The physician should have the presence of mind to initiate the radiological process for the patient. The clinician reading the X-ray images has to accurately classify this to be a potential LLD problem. The patient has to follow through the long process and has to complete the process. The relevant location or region should have an expensive X-Ray machine, with available technicians, and with available time-slots for the patient.

Typically, LLD diagnosis and detection is not a part of regular annual medical check-ups for anyone, and especially so for younger children in the age group of 5 to 12. When LLD is not identified and fixed or corrected or otherwise handled early, posture deformation, gait asymmetry, and lower-joint damages may occur in later years. Apparent LLD condition is more common than true LLD, some of the symptoms for which include: Scoliosis; Flat feet; Unleveled hips. In cases where the apparent LLD cannot be confirmed via X-rays, this proposed system and method of some embodiments is an indispensable method for accurate diagnosis.

The Applicants have further realized that some biases are prevalent and endemic in medicine. Such biases could result in deeply fallible and flawed medical diagnoses/data. This flawed data and decisions can amplify harm caused to the complex human body system. Since the initial screening is done under the experience and skill of a human practitioner, the accuracy of the first examination, which sometimes may be affected by the screener's personal condition, is the trigger for the course of action a patient/physician takes. Therefore, it is important for this to be accurate and to ‘de-bias’ via qualitative and quantitative means and via a non-biased computerized system as provided by some embodiments.

As discussed above, the analysis and classification of LLD can be complex and sometimes overlooked, even for a trained eye. This gap is addressed by some embodiments, by using Machine Learning (ML). For example, a ML unit can be configured and trained and used to perform computationally complex tasks, leading to determination or detection or determination or diagnosis or classification of certain conditions in the general population and/or in high-risk patients. Some embodiments therefore provide and use ML technologies to diagnose or detect LLD. The computational means provides consistent and reliable and non-biased first diagnosis results, without relying solely on the skills of the human screener and/or the associated problems as discussed above. This makes the system and method of some embodiments an elegant and easier and efficient solution to diagnose LLD with improved-accuracy and faster diagnosis, creating an economical long-term solution to diagnose the LLD condition across a population of patients.

Further, in the current art, individual diagnosis centers might be introducing various technologies in their own tests; which in turn renders diagnosis technology inconsistent and/or insufficiently reliable. Because each of large diagnosis centers individually utilizes its own diagnostic result data, the process can be complex, and data from multiple centers cannot be combined or aggregated by a conventional system. An electronic means to provide uniformity to the solution, according to some embodiments, removes complexities arising from such incompatibilities among medical providers and/or removes or reduces various biases. Often Artificial Intelligence (AI) unit(s) may be used for these processes, as increasingly complex diagnosis can be automated to not miss the intricate details.

An objective of some embodiments is to provide an elegant, efficient, rapid, reachable, affordable, and accurate LLD diagnosis system and method, using computational approaches such as Deep Neural Network (DNN) learning, in which diagnosis accuracy of devices can be gradually improved and dynamically updated, particularly as a greater corpus of leg images is gradually collected and analyzed. This is performed by a client-server type architecture (as a non-limiting example; or, using peer-to-peer architecture, in some embodiments, or multiple Nodes architecture) with an Internet connection, without individually modifying each of the neural network models that each end-point is currently using. The system uses Deep Neural Learning Network by which computers may think and learn like a human, continuously or constantly, and are trained to categorize or to classify objects and hence perform an Artificial Intelligence process.

Image classification techniques are used in some embodiments by or for computer vision tasks or computerized vision tasks (e.g., segmentation, object detection, and image classification), as well as pattern recognition exploiting (or utilizing) handcrafted features from a large-scale database, thus allowing the system to generate new predictions from new data and/or from existing data.

In the ML algorithm associated with some embodiments of this invention, images (or videos, or video frames) are parsed into multiple layers; and computationally higher-level features are extracted from the raw input images (or video frames). Progressively, an algorithm is trained on pre-classified images and is then validated on a separate set of pre-classified images. From the predictions that were generated on the training images, the ML algorithm compares the expected results and charts an auto correction sequence. The ML algorithm thus learns from existing data and derives a model which is then used to predict or to classify the features of new images presented to it.

In some embodiments, the ML algorithm uses Convolutional Neural Network (CNN) transforms, which apply functions such as convolution, kernel initialization, pooling, activation, padding, batch normalization, and stride to the images for processing. The CNN unit then adaptively learns various image features, and performs an image transformation, focusing only on the features that are highly predictive for a specific learning objective. Leveraging such patterns, classifiers such as sigmoid and/or SoftMax classifiers are then applied to learn the extracted and important features. This results in a Neural network model that can be used to make predictions on test or patient leg images.

The method of some embodiments involves using pipelines for LLD classification using ML techniques to develop a lightweight CNN model for automatic detection of LLD in various bilateral leg pictures or bilateral leg X-rays. This lightweight model is then adopted into the LEG-Minder devices.

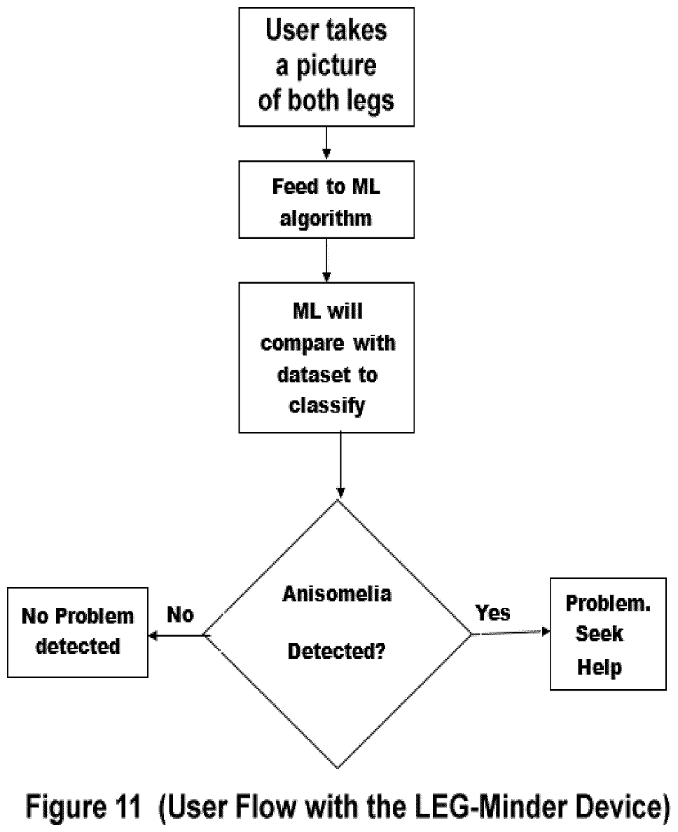

The ML models are trained using several simulated LLD image dataset with different parameters and filters in the LEGislator. With every iteration, hyperparameters are fine-tuned, activation functions are optimized to improve the accuracy of the model. Then, binary classification is employed for detection. This model is deployed in the LEG-Minder devices. Periodically, the LEG-Minder database is uploaded to the LEGislator server, which then performs an upgrade or a dynamic update of its neural network model. Then the upgraded model version is deployed or distributed again downstream, to the various LEG-Minder devices. From a LEG-Minder device standpoint, the end-user can upload a smartphone-captured or a tablet-captured (non-X-Ray, non-Ultrasound, non-CT) photograph of the legs, or (in some embodiments) a radiograph or a photograph in a bilateral fashion, and feed it to the ML algorithm, which then compares and classifies the image for LLD detection. This is summarized in the proposed user-flow as shown in FIG. 11.

The framework of some embodiments is practical and can be compared to handcrafted measurements by practitioners. The potential outcomes of some embodiments of this invention can be applied to expand into other areas of ML based classifications in the medical field, as well as specifically also measure the leg length discrepancy more accurately. Some embodiments can also predict or determine results from plain photographs (non-X-ray). For example, some embodiments may be trained to receive as input a photograph showing a group of two or more pairs of legs, of two or more persons (e.g., a class picture type of setting in a school), and to rapidly analyze it and to quickly identify potential LLD issues of a particular person (e.g., child, student) in the group photograph, and thus alert the parents or caregiver of that particular person (child, student) to seek further medical help and aid in early detection.

The proposed solution is elegant and sufficiently efficient to be applied in the context of reaching young children in underprivileged or underserved or rural or non-urban communities, by identifying LLD early via a regular smartphone-captured photograph, which can be uploaded remotely (e.g., via a parent, via a caregiver) into the LEG-Minder device with appropriate controls via a local medical practitioner, instead of getting expensive and time-consuming X-ray images which require qualified technicians to produce them and to correctly read them. Some embodiments may also help prevent expensive deformities later in the life, with a trigger to seek medical help for the necessary intervention early or at a young age. The system and method of some embodiments thus offer an elegant, rapid, efficient, affordable, reachable, and accurate LLD diagnosis system using computational non-biased approaches.

When compared to the existing clinical methods, this computational method and system of some embodiments may be more precise, and/or can take advantage of the continual learning to update the knowledge and the ML model, and is computationally more accurate than (possibly-biased or possibly erroneous) human estimation and human measurements. Inaccurate diagnosis by a human doctor can lead to higher illness burden on the patient, and well as the hospital and insurance companies. This innovation of some embodiments thus helps reduce the patient and hospital burden and costs, as well as the time and monetary resources consumed for LLD diagnoses or detection.

The fact that this algorithm can even work with non-radiological images and can use non-X-Ray photographs, or smartphone-acquired photographs, enables the use of technology even for those patients that cannot tolerate exposure to radiation of the pelvic region, or that cannot physically reach or cannot afford X-Ray imaging.

The use of non-X-Ray photographs also allows some embodiments to be used in under-served and under privileged communities, or rural or non-urban or low-income communities, where radiography reach is not available or is not affordable, or in communities in which there is a waiting line of weeks or months to schedule an X-Ray imaging visit. Some embodiments may help spread the reach of LLD diagnosis to nearly half the world's population that does not have yet access to radiological equipment.

The global radiology gap is far less discussed than infectious-disease outbreaks and natural disasters, but its dangers to public health are as urgent. In certain countries, there is a serious deprivation of radiologists and/or radiology equipment. The use of regular or non-X-Ray photographs or smartphone-captured photographs, in some embodiments, may allow the general population to use this technology that can provide more accurate and faster and cheaper diagnosis of LLD, and even junior-level or entry-level technicians may be trained to operate the system of some embodiments, obviating the need to get manual assistance from more qualified radiologists.

The time that elapses from from the point of first patient-doctor contact to the point of LLD diagnosis completion, can be significantly reduced by using some embodiments of this invention. Since there is no scheduling to be made with radiology department, patient having to come back at another time for getting X-rays, and once taken, waiting for the Radiologist to read the X-rays and then passing the information to the orthopedic doctor, the multiple steps of this process can all be cut down to a few minutes as the technician or the orthopedic doctor themselves can use the device to diagnose LLD problem in a matter of few minutes.

Some embodiments may use the following (or similar) Algorithm, which may be performed at or by the LEG-Minder client-side device and/or at the LEGislator server-side device:

Step (1): As a first step, the algorithm pre-classifies images (or discrete video frames) into a training set and a validation set.

Step (2): Next, the algorithm addresses image normalization. Since the raw input data may not already be normalized, i.e., there is no control over what pixel size a user may input, the algorithm rescales the images to normalize all the input parameters. After this step, all the images will have identical parameters although the content inside may be very different. The normalization may be performed by image re-scaling or image re-sizing; such that, for example, all raw input images are re-sized or re-scaled into normalized images in which the longest dimension of the longest leg occupies (vertically) exactly 1,200 pixels, as a non-limiting demonstrative example.

Step (3): Then, the algorithm pre-processes the image data. Image pre-processing is done by augmentation of the existing data, and also considers and takes into account overfitting the data.

Step (4) Then, the algorithm expands the scope of the input images to account for yet unseen image variations; by amending the existing images to overfit while training the data, and/or by use of various transforms like rotation, flipping, skewing, relative zoom, and other affine transformations such as translation, rotation, isotropic scaling and/or shear.

Step (5): Next, the algorithm sets or determines or selects a specific batch size, in order to process a batch of images at once; for example, 50 images per batch, or 256 images per batch or other suitable batch size. In some embodiments, the batch size is pre-configured or pre-defined; or, may be dynamically determined based on one or more pre-defined rules or criteria.

In some embodiments, the Batch Size may be dynamically configured or set or modified or updated, by the device performing the LDD classification (e.g., the LEG-Minder device and/or the LEG-islator server). In some embodiments, the Batch Size may be dynamically modified based on one or more parameters, for example: the size of each image (e.g., in kilobytes or megabytes), the cumulative size of a group of images, the size of Random Access Memory (RAM) that is available for processing, the processing resources and/or speed of the available processor or processing core or CPU or GPU, and/or other parameters. In some embodiments, the Batch Size should not be too high and not too low; and/or it should cause the same or generally the same number of images remain in every step of an epoch. In some embodiments, there is a high degree of correlation between the learning rules and the cost functions. In some embodiments, the Batch Size may also take into account the total number of images that are in the dataset or database. In some embodiments, the Batch Size should be set such that the system would not exceed the available resources (e.g., RAM memory size and/or GPU memory size); for example, some embodiments may utilize a single GPU having its own RAM memory of 6 gigabytes, and thus the Batch Size should not exceed 6 gigabytes as this is the maximum size of data that the GPU can hold in its RAM memory. A large batch size may result in faster progression in training, but does not necessarily achieve fast converging. Additionally, a small batch size may cause the training to be slow, but the converging may be fast. In some embodiments, the ML/DNN/CNN model improves with more epochs of training, and its accuracy reaches a plateau as convergence is reached. Some embodiments may utilize a process in which the system determines, within a few iterations (e.g., within not more than 5 or 8 iterations) if the algorithm would indeed converge or would not converge, based on how quick (measured by the number of iterations it takes for) the error reduction to occur and based on how fast (measured by the number of iterations it takes for) the accuracy rate to get to around 90% (or more) and to stay at approximately 95% (or more). In some embodiments, if within 5 or 6 iteration, there is already an indication that the algorithm does not converge (at all, or sufficiently fast), then the Batch Size may be dynamically modified or adjusted (increased or decreased), and the number of steps may also be adjusted, in a balanced way; such that the number of steps would equal the number of all images in the training set divided by the number of batches. In some embodiments, such implementation is harmonious to all the images in the training set; such that in each epoch, all the images are reviewed once. This approach is counter-intuitive, and the validation accuracy is utilized right after each epoch and can provide a reliable indication for whether or not the model is trending towards right direction. If the model does not appear to be efficiently converging in the desired direction within N iterations (e.g., within 5 iterations), then the system may abort the ongoing algorithm from running any further, and may modify or adjust the Batch Size and/or the number of epochs (in a balanced way as described above), and then the system restarts the algorithm with these adjusted parameters. In other embodiments, the system may automatically test the algorithm by running it for only N iterations (e.g., 5 iterations) at a Batch Size of B1 images (such as 200 images), and then repeating at a Batch Size of B2 images (such as 450 images), and then repeating at a Batch Size of B3 images (such as 700 images), and determining whether converging is expected to occur and at what rate in each such attempt, and then selecting to run the algorithm at the Batch Size (and the respective number of epochs) that appears to lead the fastest to convergence.

Step (6): Then, the algorithm invokes a binary class mode of classification, because (in some embodiments) there are exactly two possible classes for image classification, namely, either LLD exists or LLD does not exist. Optionally, in some embodiments, the system may use more than a binary class mode of classification, such as a ternary or quaternary classification, to distinguish the severity of the LLD condition (and not only the binary detection of LLD exists/LLD does-not-exist).

Step (7): Then, the algorithm passes the training image set via convolutions, to learn particular features of the training images.

Step (8): Then, the algorithm initiates pooling and image traversal through the above path of convolutions while extracting the next set of features.

Step (9): Then, the algorithm stacks multiple sets of convolutions and pooling layers as described in the prior two steps. The size of the subject image is progressively reduced, which is then fed into the dense layers.

Step (10): The algorithm also implements a SoftMax classifier (or other suitable or equivalent classifier) to reduce binary loss through the above process steps.

Step (11): The Learning rate is then adjusted for convergence to arrive at a solution.

Step (12): The algorithm then outputs a single neuron with a sigmoid activation which gives or indicates the final result on the processed image.

Step (13): Next, the algorithm performs the same process on a validation dataset to verify that the algorithm is indeed classifying images accurately.

Step (14): The algorithm is now trained, and can accept a new image for classification (namely, for LLD detection); upon which it will process the feature extraction identified earlier to finally arrive at a classification output on (or indicated by) the single neuron.

Image Pre-Processing (Step 3 in Algorithm):

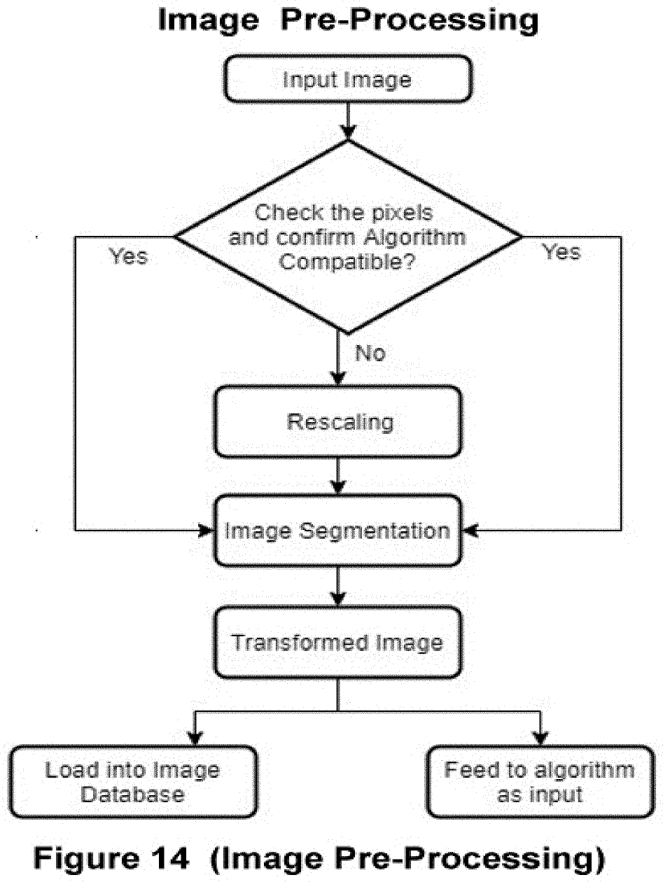

Image preprocessing refers to step 3 defined in the algorithm summary. The system can accept various image formats, such as JPEG, GIF, PNG, etc., typically used for photographic images. Formats such as DICOM, NIFTI, and Analyze AVW are used in medical imaging. Formats such as TIFF, ICS, IMS, etc., are used in microscope imaging. Image data will be stored as a mathematical matrix. Approximately, 2D image of size 1024-by-1024 pixels is stored in a matrix of the same size. It takes an image as an input and recognizes image pixels and converts it into a mathematical matrix. As shown in FIG. 14, the algorithm then checks for the image's compatibility with a predictive model. If the image is compatible, it is fed directly to the image segmentation section, skipping the rescaling. During the rescaling operation, first, a combination of linear filters and non-linear filters are used to remove the undesirable properties in the input image. Image enhancement, if needed, is accomplished either in spatial or frequency domain as necessary. In the Image Segmentation step, the image is segmented to separate the background and foreground objects. All the objects are marked with different markers setting a clear path for the predictions using the ML model. The transformed image is then stored in the database and fed to a predictive model for further processing. The Image Preprocessing flowchart shows a detailed outline of the process.

Convolutional Neural Network Layers (Steps 7, 8, and 9) in Algorithm:

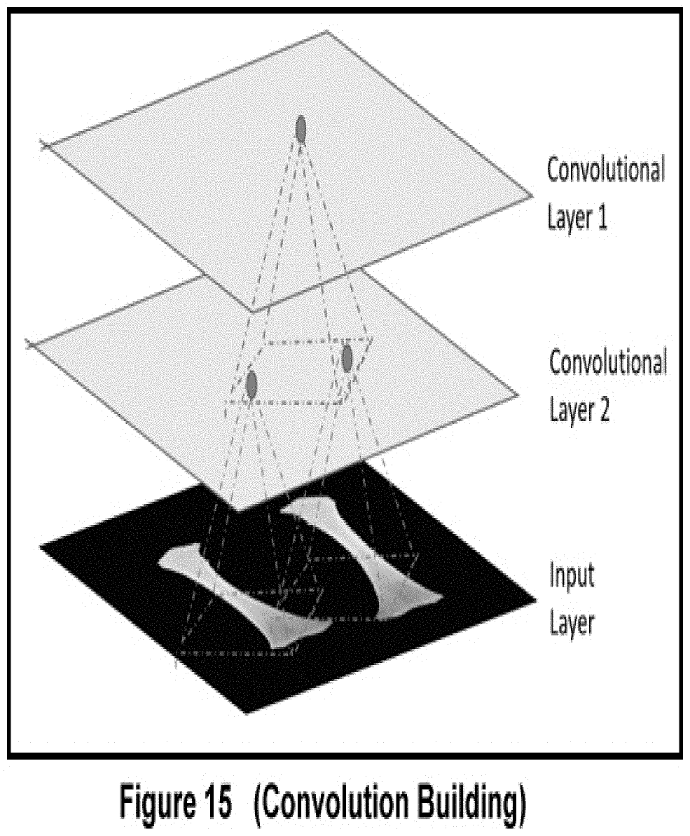

Reference is made to FIG. 15, and to steps 7, 8, and 9 in the algorithm summary. In the convolutional neural network (CNN), the neurons in the first convolutional layer are not connected to every single pixel in the input image, but only to pixels in their receptive fields. In turn, each neuron in the second convolutional layer is connected only to neurons located within a small rectangle in the first layer. This architecture is selected so that it allows the network to concentrate on small low-level features in the first hidden layer, then assemble them into larger higher-level features in the next hidden layer, etc.

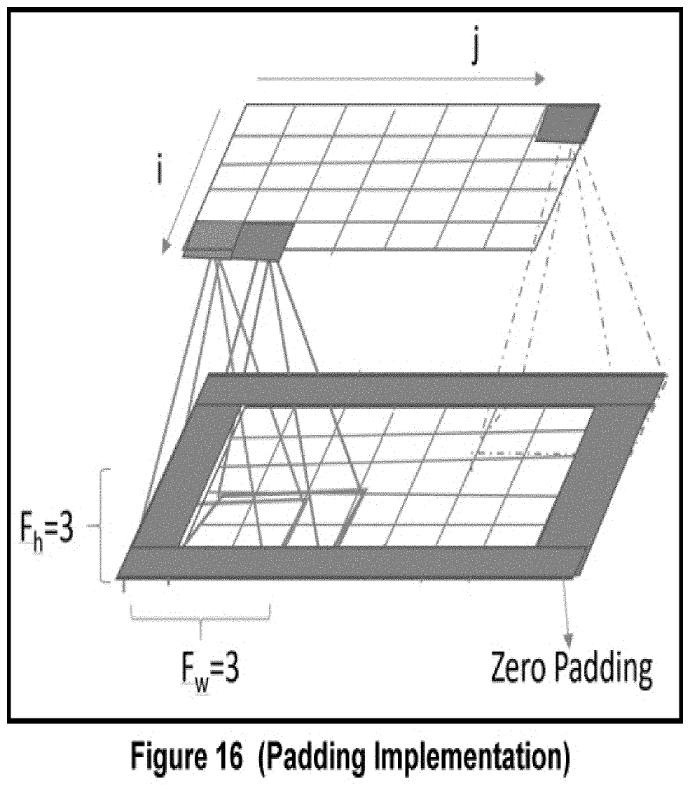

For example, a neuron located in row i, column j of a given layer is connected to the outputs of the neurons in the previous layer located in rows i to i+fₕ−1, columns j to j+f_{w}−1, where fₕ and f_{w} are the height and width of the receptive field (e.g., as shown in FIG. 16, padding implementation). Zero padding is implemented, such that a layer has the same height and width as the previous layer, by adding zeros around the inputs.

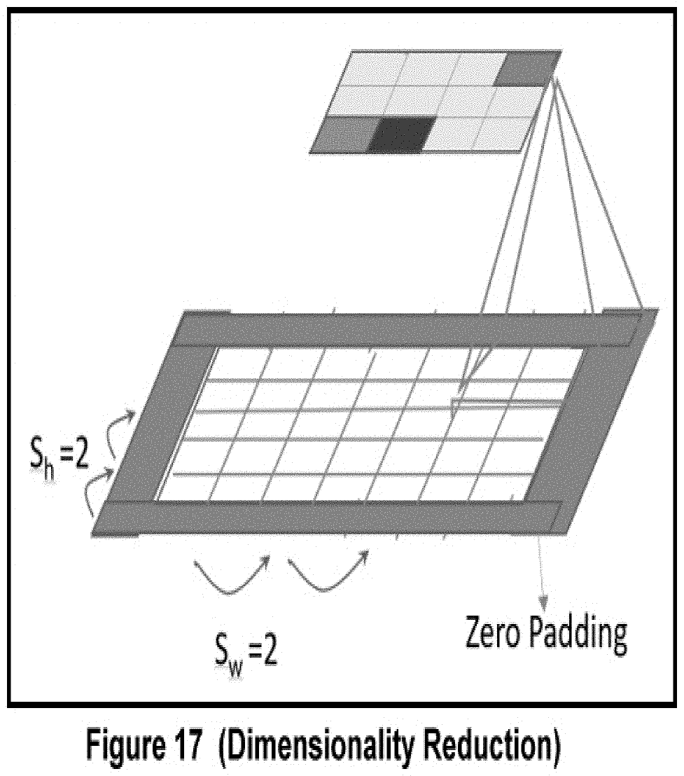

In cases where the input image layer is to be connected to a much smaller layer, a technique to space out the receptive fields is implemented so that the model's computational complexity is dramatically reduced. This is innovative because it is not a generic method to implement stride. Since there is no guarantee what the input image would look like, an illustration for a 5×7 input layer (with zero padding) to connect to a 3×4 layer, using 3×3 receptive fields and a stride of 2 is illustrated in FIG. 17 (Dimensionality Reduction). Here, the stride is the same in both directions, but it may not necessarily be so with the input images. A neuron located in row i, column j in the upper layer is connected to the outputs of the neurons in the previous layer located in rows i×sₕ to i×sₕ+fₕ−1, columns j×s_{w} to j×s_{w}+f_{w}−1, where sₕ and s_{w} are the vertical and horizontal strides.

Filters in Convolution Neural Network Layers (Steps 7, 8, 9):

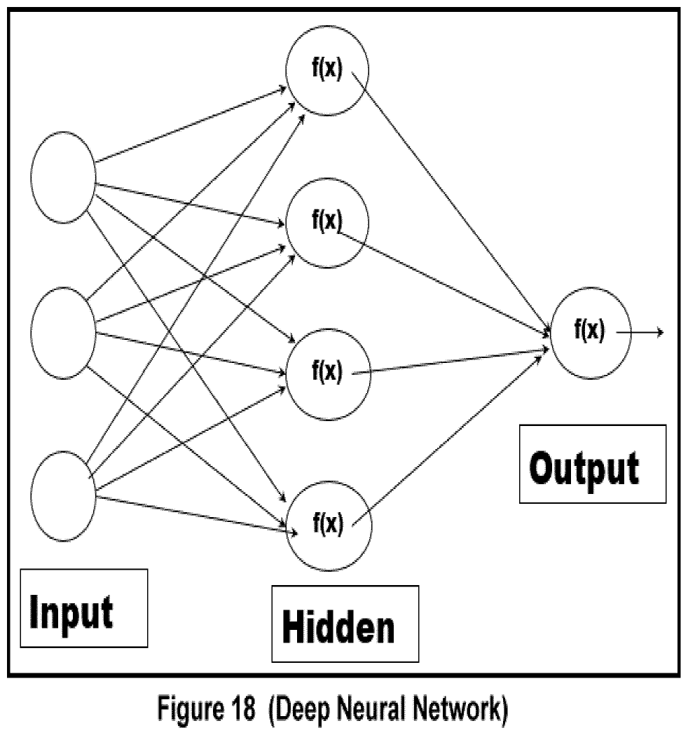

A neuron's weights, which are referred to as filters or convolution kernels, are assigned as a small image which is equal to the size of the receptive file. The first filter is a vertical filter, which is a square matrix full of Zero values, except for the central i^{th} column, of One values. The corresponding neurons will ignore everything in their receptive field except for the central vertical line. This technique ensures that the horizontal white lines get enhanced, while the rest gets blurred. The second filter is a horizontal filter, which is again a square matrix full of Zero values, except for the central j^{th} row, of One values. The corresponding neurons using these weights will ignore everything in their receptive field except for the central horizontal line. This technique ensures that the vertical white lines get enhanced while the rest gets blurred. During training of the convolutional layer, the algorithm will automatically learn the useful filters for its task of processing an image, and the layers described above will learn to combine them into more complex patterns. This allows the algorithm to stack such filters. Such combination of filters will be inputs in each convolution, and the output is one feature map per filter. It has one neuron per pixel in each feature map, and all neurons within a given feature map share the same parameters. Neurons in different feature maps use different parameters. Thus, a convolutional layer simultaneously applies multiple trainable filters to its inputs, making it capable of detecting multiple features anywhere in its inputs. All neurons in a feature map share the same parameters, thus dramatically reducing the number of parameters in the model. Once the CNN has learned to recognize a pattern in one location of the image, it can recognize that pattern also in any other location within the image. Sometimes, in case of normal human leg photographs, images are composed of multiple sublayers: one per color channel. This case is illustrated in FIG. 18, demonstrating a Regular photograph with three colors. At a basic level, there are red, green, and blue (RGB) values (or channels), while grayscale images have just one channel. When some of the latest photography techniques are used, some images may also have extra light frequencies (such as infrared).

In this case, a neuron located in row i column j of the feature map kin a given convolutional layer l, is connected to the outputs of the neurons in the previous layer (l−1), located in the rows (i×Sₕ) to (i×sₕ+fₕ−1) and columns (j×s_{w}) to (j×s_{w}+f_{w}−1), across all feature maps (in layer l−1).

In order to compute the output of a given neuron in a convolutional layer, the following formula may be used:

$${z}_{i , j , k} = {b}_{k} + {\sum }_{u = 0}^{{f}_{h} - 1} {\sum }_{v = 0}^{{f}_{w} - 1} {\sum }_{{k}^{′} = 0}^{{f}_{n ′} - 1} {x}_{i ′ , j ′ , k ′} \times {w}_{u , v , k ′ , k} with \{ \begin{matrix}{i}^{′} = i \times {s}_{h} + u \\ {j}^{′} = j \times {s}_{w} + v\end{matrix}$$

In this equation:

z_{i,j,k} is the output of the neuron located in row i, column j in feature map k of the convolutional layer (layer l).

sₕ and s_{w} are the vertical and horizontal strides, fₕ and f_{w} are the height and width of the receptive field, and fₙ, is the number of feature maps in the previous layer (layer l−1).

x_{i,j,k} is the output of the neuron located in layer l−1, row i′, column j′, feature map k′.

bₖ tweaks the overall brightness of the feature map k (in layer l).

w_{u,v,k′,k} is the connection weight between any neuron in feature map k of the layer l and its input located at row u, column v and feature map k′.

Pooling Layers (Steps 7, 8, 9 in Algorithm):

Pooling layers shrink the input image in order to reduce the computational load, the memory usage, and the number of parameters and this is specifically done to reduce the possibility of overfitting. Each neuron in a pooling layer is connected to the outputs of a limited number of neurons in the previous layer, located within a small rectangular receptive field. Its size, the stride, and the padding is defined, but the pooling neuron will be assigned no weights; all it does is aggregate the inputs using an aggregation function, such as the max or mean. Only the max input value in each receptive field makes it to (or, is transferred to) the next layer, while the other inputs are dropped or discarded. At the end of this step, the image still looks generally identical or generally similar to the input image, but the pixel density is drastically reduced. This reduces the computations, memory usage, and the number of parameters. This stage may offer a small amount of rotational invariance and a slight scale invariance. This invariance is useful in cases where the prediction should not depend on these details for the classification task.

The Applicants have realized that in some situations, Underfitting occurs when a model cannot adequately capture the underlying structure of the data. An under-fitted model is a model where some parameters or terms that would appear in a correctly specified model are missing. Under-fitting may occur, for example, when fitting a linear model to non-linear data. Such a model will tend to have poor predictive performance. Generally, realized the Applicants, over-fitting may exist because the criterion used for selecting the model is not the same as the criterion used to judge the suitability of a mode, i.e., a model might be selected by maximizing its performance on some set of training data, and yet its suitability might be determined by its ability to perform well on unseen data; then over-fitting occurs when a model begins to “memorize” training data rather than “learning” to generalize from a trend. For instance, if the number of parameters is the same as or greater than the number of observations, then a model may be able to perfectly predict the training data simply by “memorizing” the data in its entirety. Such a model, though, would typically fail severely when making predictions. The potential for overfitting, realized the Applicants, depends not only on the number of parameters and data, but also on the conformability of the model structure with the data shape, and/or on the magnitude of model error compared to the expected level of noise or error in the data. Even when the fitted model does not have an excessive number of parameters, realized the Applicants, it may be expected that the fitted relationship will appear to perform less well on a new data set than on the data set used for fitting. To lessen the chance or amount of overfitting, in accordance with some embodiments, one or more techniques may be used (e.g., model comparison, cross-validation, regularization, early stopping, pruning, Bayesian priors, dropout). The basis of some techniques is either (1) to explicitly penalize overly complex models, or (2) to test the model's ability to generalize by evaluating its performance on a set of data that was not used for training, which is assumed to approximate the typical unseen data that a model will encounter. Some embodiments may thus use a combination of these techniques mentioned above, including for example: to prune the data first by applying filters on images, and to perform cross-validation and regularization.

Similarly, realized the Applicants, a model with too little capacity cannot adequately learn the problem, whereas a model with too much capacity can learn it too well or excessively and thus overfit the training dataset. Both cases may result in a model that does not generalize well. To address this issue, some embodiments are configured to reduce generalization error so that they can still use a larger model by introducing regularization during training that keeps the individual weights of the model small. This technique reduces overfitting, and/or leads to faster optimization of the model and/or better overall performance. Sometimes the system may not have all the data needed to come up with the necessary parameters needed. This is referred to as underfitting. Underfitting is the inverse of overfitting, meaning that the statistical model or machine learning algorithm is too simplistic to accurately represent the data. A sign of underfitting, realized the Applicants, is that there is a high bias and low variance detected in the current model or algorithm used (the inverse of overfitting, which is characterized by low bias and high variance). This can be gathered or deduced from the Bias-variance tradeoff, which is a method of analyzing a model or algorithm for bias error, variance error, and irreducible error. With a high bias and low variance, the result of the model is that it will inaccurately represent the data points, and thus insufficiently be able to predict future data results. Indeed, some embodiments may utilize a rule that an underfitted model may ignore some important replicable (i.e., conceptually replicable in most other samples) structure in the data and thus fail to identify effects that were actually supported by the data. Some embodiments may address this, for example, by increasing the capacity of the network and/or by increasing the size of the image data that is available for training the ML model and/or for dynamically updating and optimizing it.

in some embodiments, a few convolutional layers are stacked, and each one is followed by a rectified linear activation function or ReLU. This is a piecewise linear function that is configured to output the input directly if it is positive; otherwise, it will output zero. This ReLu is used to achieve better performance. In some embodiments, the stacking of layers may be, for example: The ReLU layer, then a pooling layer, then another few convolutional layers again followed by ReLU, then another pooling layer. The input image becomes smaller and smaller as it progresses through the network and its layers, but it also typically gets deeper with more feature maps. At the top of the stack of layers, a regular feedforward neural network is added, with a few fully connected layers followed by ReLU and the final layer outputs the prediction.

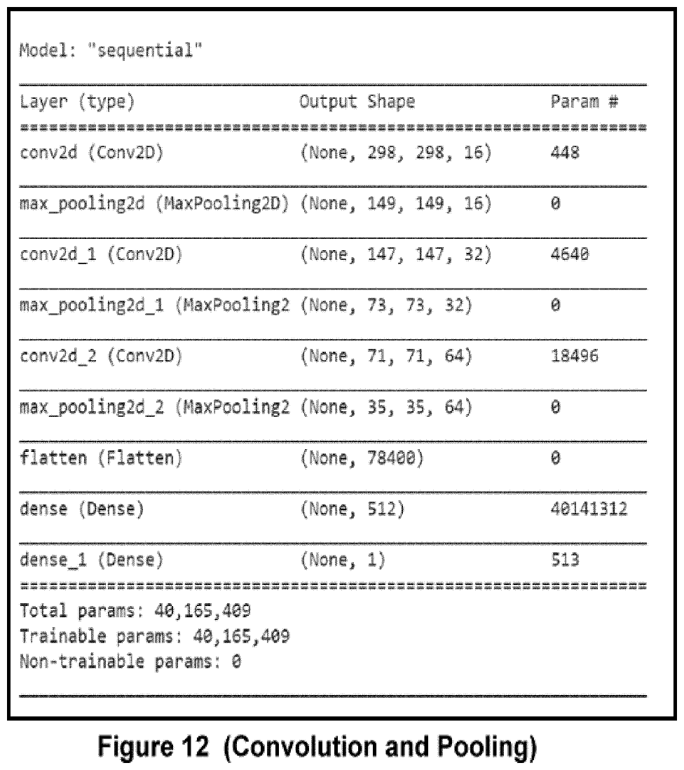

FIG. 12 shows a demonstrative example of the convolutional blocks, with a depth of several layers, demonstrating a set of convolutions followed by pooling. The input image is (for example) 300 by 300 pixels. There is a single neuron with a sigmoid activation on the output. The summary of the layers is also shown with the corresponding size changes. The first convolution reduces that to 147 by 147 pixels. From there, convolution loop repeats until the image size is reduced to 35 by 35 pixels, which is then fed into the dense layers. In a demonstrative example, a total of 40,165,409 trainable parameters were identified with this algorithm iteration according to some embodiments.

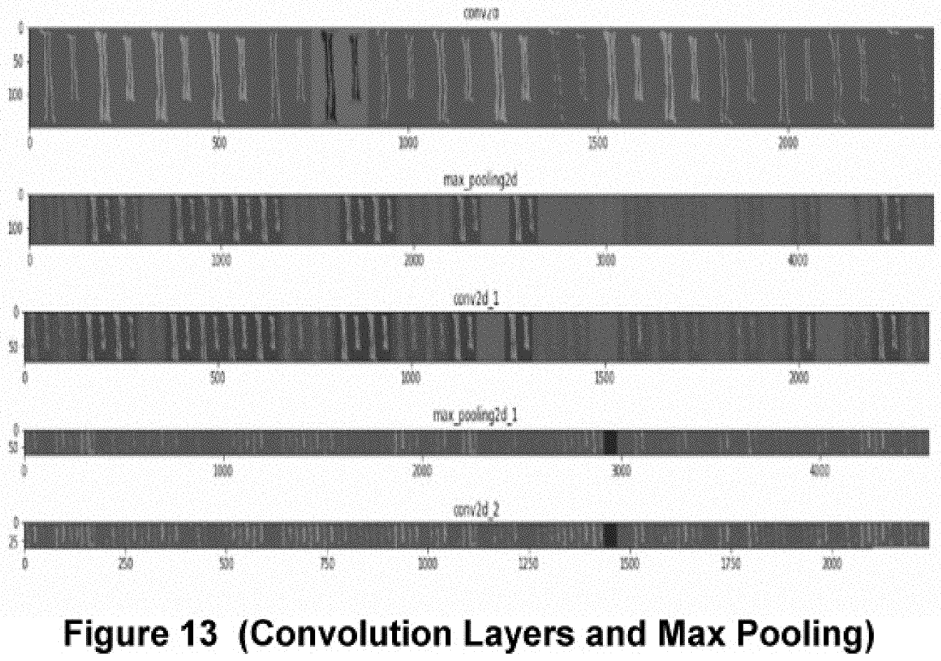

While it may be difficult to examine a CNN on a layer-by-layer basis, each layer's output may be visualized and extracted features may be seen, as demonstrated in FIG. 13.

Referring now to the SoftMax Classifier (Step 10 in the algorithm): the SoftMax Regression classifier is used to predict only one class at a time. Even though it is generally used for multiclass (since the outputs are strictly limited to mutually exclusive classes), the Applicants have realized that counter-intuitively this classification works well for a clear, unequivocal classification in accordance with some embodiments. Overall, the model is aimed at estimating probabilities and making predictions. The objective for the algorithm is to overall estimate a high probability ‘p’ for the intended class, and consequentially a low probability ‘(1−p)’ for the other class. This is accomplished by minimizing the cost function for cross entropy. The cross entropy is structured for measuring how well the estimated class probabilities matches the target class, by “penalizing” the model when it estimates a low probability for a target class. The Cross entropy cost function may be represented by the following expression:

$$J ( θ ) = - \frac{1}{m} {\sum }_{i = 1}^{m} {\sum }_{k = 1}^{K} {y}_{k}^{( i )} log ( {\hat{p}}_{k}^{( i )} )$$

In this equation:

yₖ⁽ⁱ⁾ is the target probability that the i^{th} instance belongs to class k. Since the prediction is either Yes or No, it will be a 1 or a 0 depending on whether the instance belongs to the class or not. If the assumptions are wrong, the cross entropy will be greater by an amount called the Kullback-Leibler (KL) divergence. The cross entropy in such cases, will be governed by:

H(p,q)=−Σₓp(x)log q(x)

Where p and q represent the discrete probability distributions.

The gradient vector of this cost function with regard to θ^{(k)} is:

$${∇}_{{θ}^{k}} J ( θ ) = \frac{1}{m} {\sum }_{1}^{m} ( ( {\hat{p}}_{k}^{( i )} - {y}_{k}^{( i )} ) {X}^{( i )}$$

With this, the gradient vector for every class is computed, and then Gradient Descent is used to find the parameter matrix Θ that minimizes the cost function determined by the cost entropy cost function.

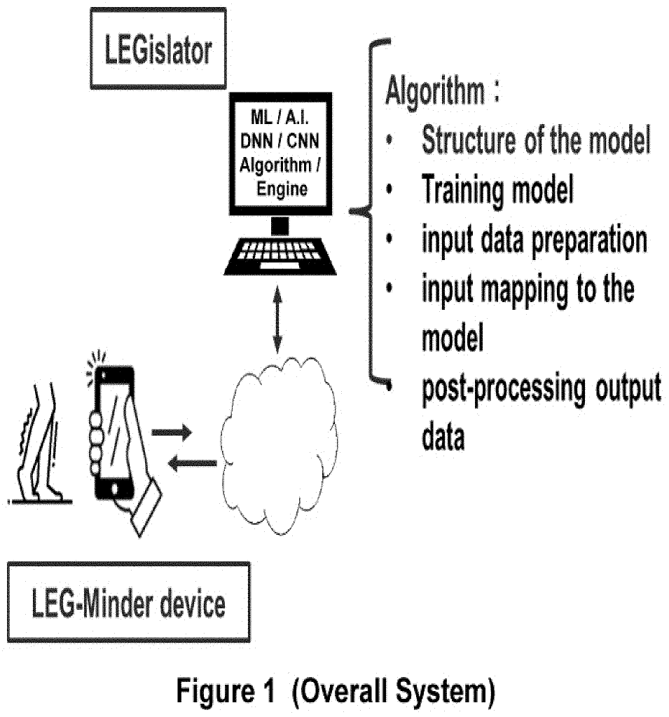

Reference is made to FIG. 1, demonstrating the overall system including LEG-Minder and LEGislator components for the Leg Length Discrepancy (LLD) diagnosis, using Deep Neural Network (DNN), according to some demonstrative embodiments of the present invention.

In some embodiments, a Deep Neural Network (DNN) Implementation may be used. In order to non-linearly combine information in the server, Dense/Deep Neural Networks may be used. They are used in the server ‘LEGislator’ device. The “LEGislator’ device can be used on its own to also make categorical predictions, although in some embodiments it is primarily used to improve the CNN's accuracy in the LEGMinder device (the client-side device, which may be located at a remote medical office or physician's office or at another end-user). Dense layers are on the server, and they are hierarchically on top of the CNN architecture in the LEGMinder devices. This allows recombination of the information learned by the convolutional layers from the client-side devices. This comprises of one passthrough input layer, one or more hidden layers, and the one final layer called the output layer. This is depicted and demonstrated in FIG. 18 showing a demonstrative Deep Neural Network. The layers close to the input layer are referred to as the lower layers, and the layers close to the outputs are referred to as the upper layers.

The main components of the algorithm are:

(a) Algorithm in the server will handle one mini-batch at a time, and goes through the full training set multiple times. Each such pass is referred to in this specification as an Epoch.

(b) Each mini-batch is passed to the network's input layer, which sends it to the first hidden layer.

(c) The algorithm then computes the output of all the neurons in this layer for every Epoch.

(d) The result is passed on to the next layer, its output is computed and passed to the next layer, and so on until it reaches the output layer. This is the forward pass: it is exactly like making predictions in the CNN, except all intermediate results are preserved.

(d) Next, the algorithm measures the network's output error.

(e) Then it computes how much each output connection contributed to the error using chain rule.

(f) The algorithm then measures how much of these error contributions came from each connection in the layer below all the way to the input layer. This will be done by propagating the error gradient backward through the network.

(g) Next, Gradient Descent is performed to tweak all the connection weights in the network, using the error gradients just computed.

(h) The Rectified Linear Unit function ReLU(z)=max(0, z) may be used for this algorithm.

Referring to FIG. 1, the individual components comprise of a “LEGislator” based on ML which is connected to the Internet and performs DNN learning on the neural network of the “LEG-Minder” device. In particular, some embodiments provide a system in which patient leg photos and/or diagnostic result data are acquired in each diagnosis center (e.g., medical office, physician's office, healthcare center) by the LEG-Minder device and are then uploaded (or copied or sent upstream to a central server) to the neural network learning server “LEGislator”. The central learning server performs DNN learning, and updates the neural network model; which in turn is installed in the ‘LEG-Minder’ of the diagnosis center via an update cycle or an update distribution cycle (e.g., every hour, or every day, or every week).

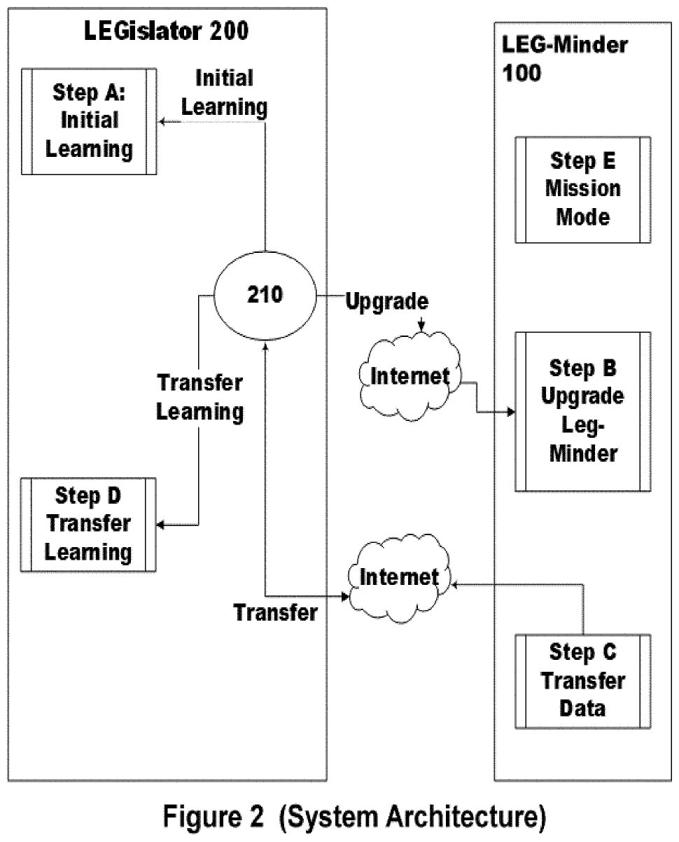

FIG. 2 is a block diagram of the LEG-Minder and the Neural Network Server “LEGislator” combination, according to some demonstrative embodiments of the present invention. This is a system level architecture representation, showing the individual actions/functions that each of the devices may be configured to perform.

Leg-Minder Device (100):

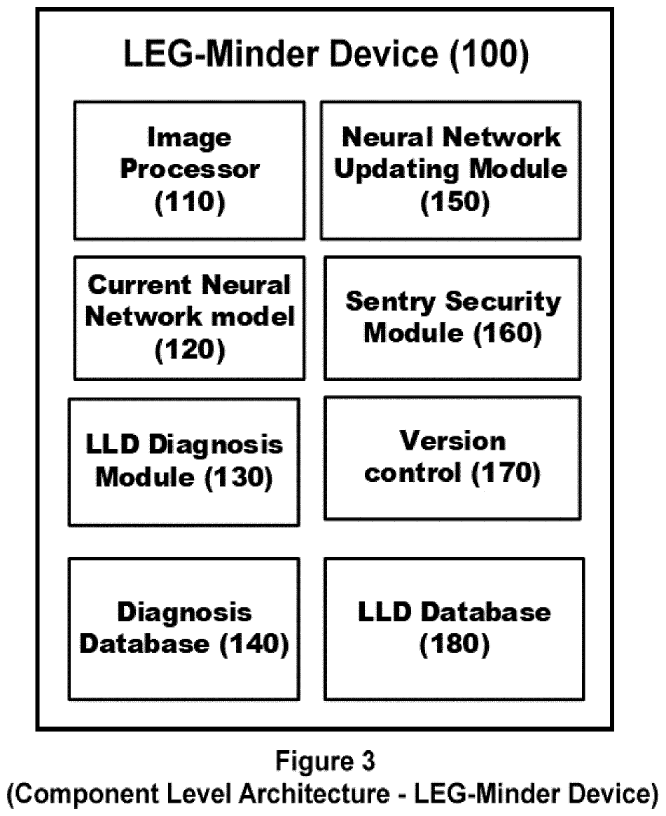

FIG. 3 demonstrates a Component Level architecture of the LEG-Minder Device (100). This device captures the input data from the patient, and is equipped with a neural network model that runs on a local Machine Learning engine, which may be implemented as computer software and/or using hardware components (e.g., processor, memory unit, storage unit).

The LEG-Minder device (100) captures the patient image, directly through a camera and/or by allowing a user to upload image (e,g., photograph, X-ray image) to the LEG-Minder device using external means, such that the image(s) are received into the image processor module (110). The neural network model, which is initially installed in the LEG-Minder device (100), may be referred to as the “current neural network model” (120), and it may be updated and modified and optimized from time to time or on a continuous basis. Further, “No-LLD” or “LLD Not Detected” refers to a photograph or X-ray image of a person that is classified Not to have Anisomelia or Leg Length Discrepancy; whereas “LLD” or “LLD exists” or “LLD Detected” photograph or image represents one of a non-ignorable possibility of Leg Length Discrepancy and possibly requiring further examination by a specialist Orthopedic for treatment/rectification. The image is processed to find if the subject picture has LLD or Not, by using the LLD Diagnosis module (130). When a new image is diagnosed or processed by the LLD diagnosis module (130), the corresponding computation result, as to whether the subject image has LLD or not, is stored in the classified Diagnosis Database (140).

Periodically, the LEG-Minder device transfers its local Diagnosis Database (140) of locally-obtained diagnosis results to the central LEGislator server, though the Neural Network Updating module (150); such as, over the Internet, over a secure channel (HTTPS), over Wi-Fi, over a cellular connection, over a wired connection, and/or by other communication means. The Neural Network updating module (150) also receives (e.g., periodically) the updated model that is distributed from the LEGislator device (200), and updates the current neural network model (120) that runs on the local LEG-minder device. As the neural network updating module (150) updates the current neural network model (120), the version history is maintained in the “Version Control Module” (170).

There are two databases in the device, namely, the LLD database for storage of raw images (180), and a diagnosis database (140) storing images that were already classified by the model.

Sentry Security module (160) ensures the integrity of the learned model, as well as governs the security aspects related to sentry operations; such as, fending off any malicious attempts to induce bad data either at the network level or at the image ingress level for the LEG-Minder device (100), and/or otherwise blocking or detecting or preventing malicious attacks on the system.

Learning Server—“LEGislator” (200):

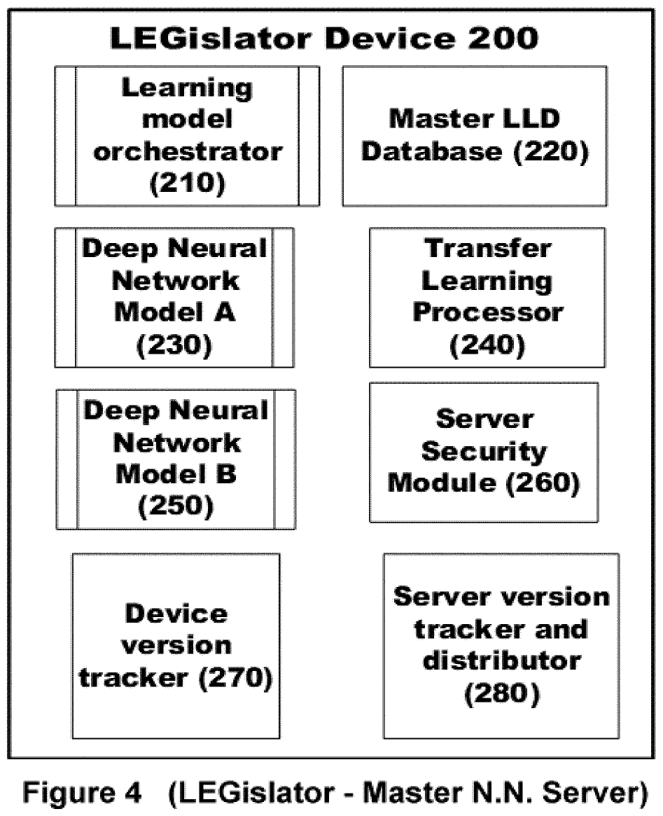

FIG. 4 demonstrates a LEGislator Master Neural Network Server, or the “LEGislator Device” (200). The function of the LEGislator Device is to perform transfer learning on the accumulated dataset, and to generate an upgraded or updated or modified neural network model to be disseminated or distributed (e.g., periodically) to the various LEG-Minder (100) device(s). The operations of LEGislator Device (200) are orchestrated or controlled by the Learning model orchestrator (210) or control unit.

Upon initiation by the Learning model orchestrator (210), the various LEG-Minder device(s) (100) or at least some of them will transfer their diagnosis database (140) to the Master LLD database (220). The transfer learning processor (240) then uses Deep Neural Network Model A (230) and the Master LLD database (220) to perform deep learning using DNN techniques to generate a Deep Neural Network Model B (250).

Deep Neural Network Model B (250) reflects all the current learning and all the up-to-date aggregated data in this client server architecture. The Server version tracker and distributor (280) keeps track of Deep Neural network model A (230) and the Deep Neural network model B (250). It performs a switch to model B from the previous model which was model A, at the appropriate time; as well as performs dissemination or distribution or copying of the current model (model B) to all the LEG-Minder devices (100) via the Internet or via a secure communication channel (e.g., HTTPS), thus performing an auto-upgrade and a dynamic/periodic update of the model utilized by LEG-Minder devices. However, to prevent any spurious devices from getting updates and/or to ensure that no compromised LEG-Minder device (100) ever gets the update, the Server security module (260) ensures that proper Authentication, Authorization, Accounting and Auditing is conducted. For this purpose, Server security module (260) will work in conjunction with the Device version tracker (270). The Device version tracker (270) is a database that keeps track of every device that connects to the neural network, its associated credentials and access privileges and security parameters, to ensure integrity and security of the overall system.

After updating to the latest neural network model, the LEG-Minder device updates its Version control (170). Depending on certain pre-defined rules or constraints, the Version control (170) may choose to accept or reject the downloaded version from the LEGislator device. For example, a particular LEG-minder device may download Model Version 18; may run it for several iterations, and may observe that this model version is extremely slow to converge and/or to yield classifications prediction, such as 20 times slower than Model Version 17, due to one or more reasons which may be known or not known (e.g., due to Model Version 18 being computationally complicated for the processing resources/memory resources of this specific LEG-minder device); and thus, this LEG-minder device may autonomously determine that the most-recent model version is not suitable and/or is not efficient for the particular resources or configuration or constraints of this device, and its own Version Control unit may revert to the previous version, which was Model Version 17; and may optionally send a report or a message to the LEGislator server, to report that the new Model Version 18 is not suitable for this particular LEG-minder device.

System Architecture:

At a system level, the LEGislator server and a plurality of the LEG-Minder devices form a client-server architecture; the LEG-Minder device is the client, and the LEGislator is the server. This is depicted in FIG. 2 showing System Level Architecture, as a non-limiting example; other suitable architectures may be used, for example, peer-to-peer architecture, or an architecture having a plurality of Serving Nodes that serve batches or groups of Client Devices.

In a demonstrative example, consider a healthcare network which is present in multiple locations and across multiple states/cities within the USA. Each medical practice or medical office or healthcare facility can cater to a certain number of patients or to a particular population, typically around or near its geographic location. In the case of an orthopedic doctor, when the doctor sees a range of patients, that particular doctor develops a certain level of knowledge and expertise. Given the regionalities, population density belonging to a certain ethic origin or geographic location, may see (e.g., more often) a certain type of patients, and may become experts within that population segment and know better what to expect. This is based on the specialist's ‘learning’. Boston or Miami may have a different set of patients who bring their own nuances. So, the “LEGminder” operates as a machine in a way which may emulate the expertise of a regional doctor with regard to a local or regional population. The plurality of the devices is emulating many such local or regional doctors, who get their own local or regional learnings. They get their learning based on the patients they see, which in turn is typically a function of their geographical location; since a doctor (or a LEG-minder device) in Miami would typically see patients who reside in Florida, and not in California.

In the above example, the system of some embodiments allows to replace the regional doctors with one doctor who serves the entire humanity or the entire global population or the entire U.S. population, and becomes an expert (via machine learning) due to accumulated regional/local knowledge. Because the server would get images from a vast number of patients that are scattered all over the USA or all over the world, the server's knowledge base would be huge, and it may be able to generate learned insights and a model that cannot be achieved by a local/regional facility by itself. This is a direct relationship with the number of patients they see. So, the knowledge has a direct correlation with the “learning” that can be obtained. In this case, we have a server device, that takes the regional leanings and builds and also dynamically updates a master database, keeping track of the individual leanings (similar to a journal of regional doctors). In the case described above, we would rather see the entire network provide a similar experience to the patients. For this to happen, every device on the network is configured to be working based on the same unified learning that was derived from all the accumulated knowledge. Therefore, the server aggregates the learning, develops a common and unified and aggregated base from which each client device would operate, and serves this information to the individual client devices.

Since the LEG-Minder device is a machine, it needs security measures. Hence the security aspects are embedded to prevent someone from providing pictures of donkey's legs or animal legs or non-human legs versus the expected human legs, for example.

As shown in FIG. 2, the Learning model orchestrator (210) controls the operations of the LEGislator 200. The Learning model orchestrator (210) performs four fundamental operations. Those are: Initial Learning; Transfer Learning; Database Transfer; Upgrade.

As shown in FIG. 2, the LEG-Minder 100 performs three fundamental operations. They are: Mission mode (Learn and Predict); Upgrade; Transfer database.

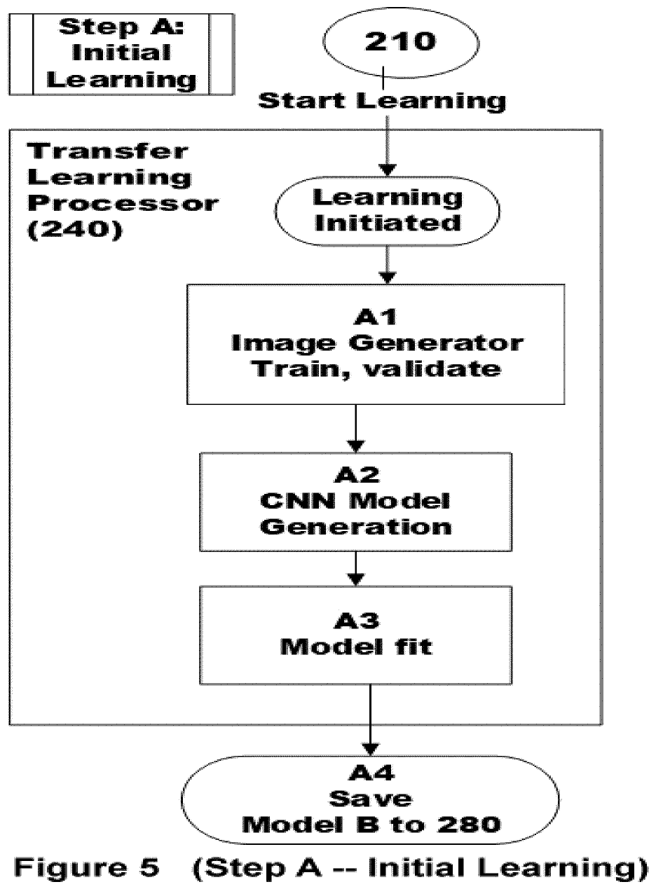

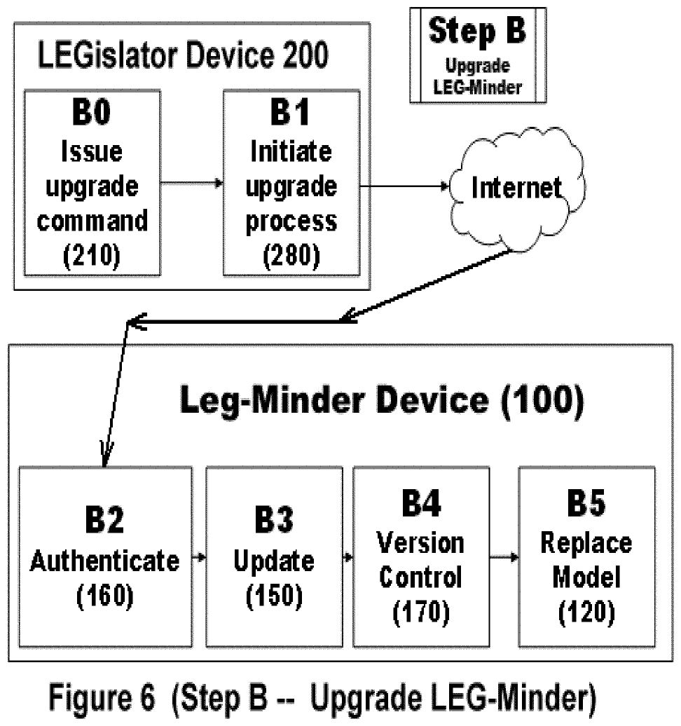

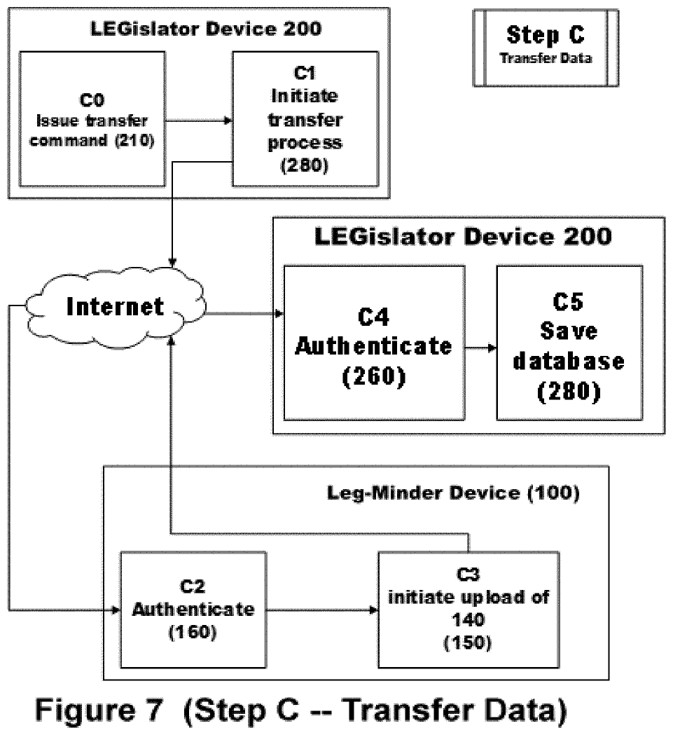

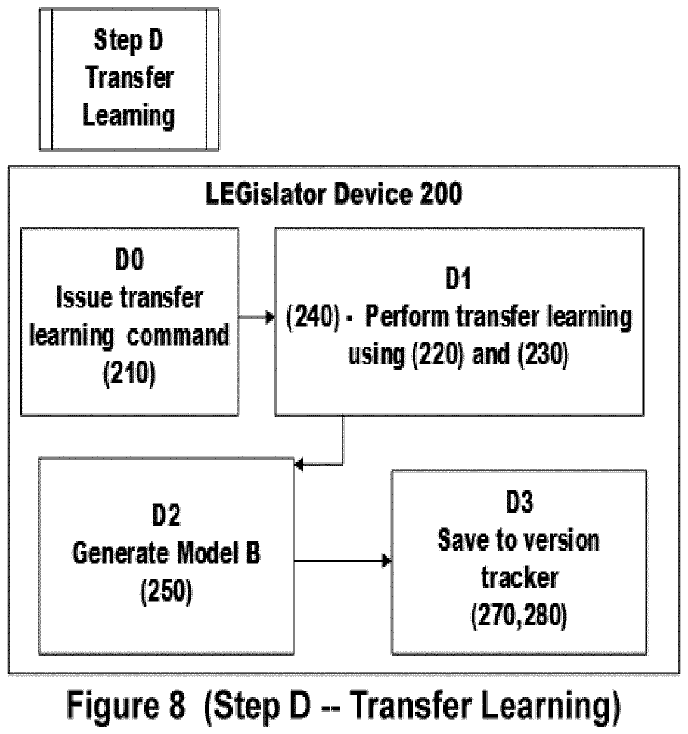

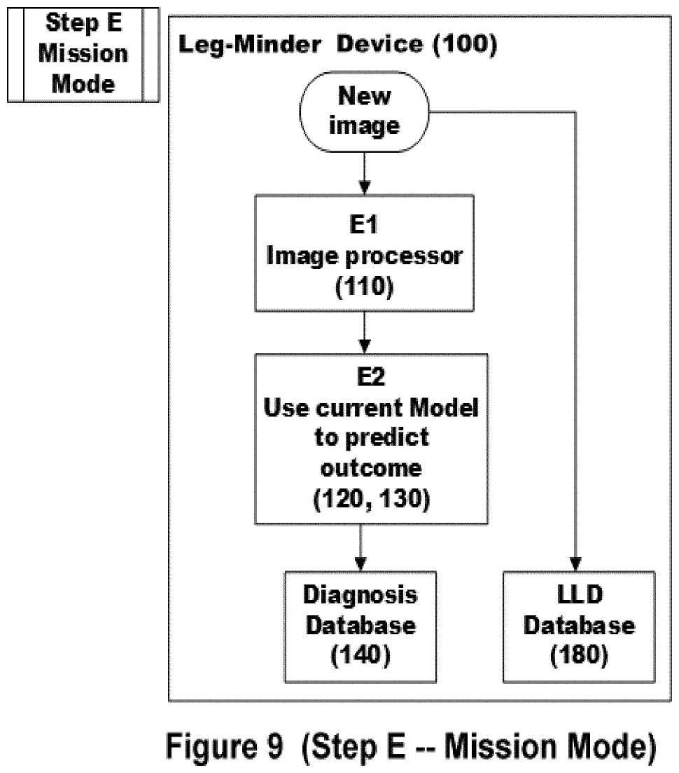

Each of the operations mentioned above are described in greater detail in—FIG. 5, FIG. 6, FIG. 7, FIG. 8 and FIG. 9 each process step described in detail as below.

Step A—Initial Learning

As shown in FIG. 5, upon receiving the trigger or a signal or a command from the learning model orchestrator (210), the Transfer learning processor (240) will perform:

STEP A1: Initiate a learning sequence with its associated image generators, and create the train and validation datasets as described in the Algorithm Summary.

STEP A2: Generate the CNN model, by stacking multiple sets of convolutions and pooling layers along with the dense layers, and by applying a SoftMax classifier.

STEP A3: Adjust the model fit, by modifying or setting the value of adjustable parameters, such as the learning rate.

STEP A4: Save the resulting Neural Network model “B” in the server version tracker and distributor (280).

Step B—Upgrade the LEG-Minder devices

As outlined in FIG. 6, Step B—Upgrade the LEG-Minder devices, the output of STEP A above is used to update dynamically and/or periodically the LEG-Minder (100) device(s). Steps associated with this are as described below with reference to FIG. 6:

STEP B0: The LEGislator device (200) will, upon initiation or signal or command from the learning model orchestrator (210), initiate the upgrade command or process to LEG-Minder device(s) (100).

STEP B1: The upgrade process is initiated by the Server version tracker and distributor (280) over an Internet connection and/or via a secure communication channel (e.g., over HTTPS or other encrypted channel).

STEP B2: The LEG-Minder device (100) receives the command into the Sentry Security module, and authenticates the command received.

STEP B3: Upon authentication, the Neural Network updating module (150) will update the current neural network model sent by (or, obtained from, or downloadable from) the LEGislator device (200) which operates as a server.

STEP B4: Upon successful update verification by Neural Network updating module (150), the version control module (170) will update the version number.

STEP B5: The current neural network model (120) is then replaced with the newly downloaded updated model or up-to-date model.

Step C: Transfer Data

At this point, the LEG-Minder device has acquired all the new learnings from the server, and continues to diagnose LLD and continues to update its local database as described in the component section. To provide the new learnings (that were obtained locally) back to the LEGislator central server, the following steps are used as shown in FIG. 7:

STEP C0: A transfer command is issued by the learning model orchestrator (210) of the LEGislator device (200)

STEP C1: A command is issued to initiate the transfer process by the Server version tracker and distributor (280) over an Internet connection (or a secure communication channel) to a specific LEG-Minder device (100).

STEP C2: The Sentry Security module (160) receives it, authenticates that it is intended for the correct LEG-Minder device (100), and then relays it to the Neural Network Updating module (150).

STEP C3: The Neural Network Updating module (150) initiates an upload of the local diagnosis database (140) to the LEGislator (200) central server, via the Internet or via a secure communication channel.

STEP C4: The Server security module (260) ensures that proper Authentication, Authorization, Accounting and Auditing are employed, and relays the relevant commands to the Server version tracker and distributor (280).

STEP C5: The Server version tracker and distributor (280) then saves the uploaded images to the Master LLD database (220), thereby incorporating into the central master database the additional data that was provided by the scattered plurality of LEG-minder devices.

This will initiate or trigger the transfer learning process in the LEGislator (200) server, as shown in FIG. 8.

Step D—Transfer Learning:

As shown in FIG. 8, STEP D0 includes: A transfer learning command is issued by the learning model orchestrator (210) of the LEGislator device (200)

STEP D1: The transfer learning processor (240) then uses Deep Neural Network Model A (230) and the Master LLD database (220), to perform deep learning using DNN techniques.

STEP D2: The above steps results in the generation of the Deep Neural Network Model B (250).

STEP D3: The updated network model (model B) is saved in Device version tracker (270) along with the credentials of the LEG-Minder (100) and the Server version tracker and distributor (280).

Step E—Mission Mode:

When the LEG-Minder Device (100) is in Mission mode, as shown in FIG. 9, it is ready of diagnosis. When a new image is presented to the LEG-Minder Device (100) for diagnosis of LLD, it performs the following steps:

STEP E1: The image processor (110) processes the image per the algorithm described under Algorithm Summary.

STEP E2: Using the current neural network model (120) and the LLD Diagnosis module (130) to process the image, and using the most up-to-date model that is currently installed in the LEG-minder device, it makes a prediction and then updates the local Diagnosis database (140) and the LLD database (180).

A demonstrative model to implement the bone measurement:

Some embodiments may utilize an innovative algorithm to find the difference in length of two bones, such as in pixels in a radiographic image of two legs. This may be done with the assistance of several different image processing libraries, such as OpenCV. The Applicants have realized that sometimes, the bones may be rotated at an angle of 5 or 6 degrees (e.g., relative to each other), which makes finding the minima and maxima of each bone more difficult and may require particular processing to compensate for such slanting or angulation.

In some embodiments, the following steps may be taken to find the LLD of a Tibia: Converting the image to grayscale; Apply Laplacian Edge Detection so that the edges are highlighted and the bone itself is darker (this makes finding contours simpler); Use cv2's findContours( ) method and get the two largest contours found in the image; Surround this contours with rectangles; Rotate each of these contours so that the width of rectangle is perfectly level; Find top extreme of bone; Dilate each rotated contour; Apply Laplacian Edge Detection like in step 2 above; Use cv2's find Contours( ); Find the highest location in the largest contour return by findContours( ); Return that location; Find bottom extreme of bone; Ignore side of bone because we want to detect a specific point on the bottom side; Dilate each edited and rotated contour; Apply Laplacian Edge Detection like in step 2 above; Use cv2's find Contours( ); Find the lowest location in the largest contour return by findContours( ); Modify that location's x-coordinate to account for the cutoff that may have been made in part a; Return that location; With both sets of minima/maxima, use the distance formula to compute LLD.

In some embodiments, the Differences for Computing LLD of Tibia vs Femur may be as follows: Computing LLD of femur bones is a process in which there are no minima or maxima that need to be ignored. In the case of the tibia, x-rays often include the fibula, which dips lower than the tibia and will be considered the minima when running findContours( ). To combat this, we ignored the fibula in each set of bones. The x-rays of femurs require no such modification, so the minima and maxima can be computed.

As a preliminary means of computing the LLD of x-ray images, several preprocessing steps can be taken to ensure that the algorithm has the highest accuracy in recognizing the appropriate minima and maxima of each leg. When considering an x-ray image of bones, the complications are manifold: the contours of each bone must be identified, the algorithm must account for each bone rotated at independent angles, and surrounding bones need to be ignored. This may require a series of image transformations and rotations which will effectively emphasize edges and deemphasize clusters of similarly colored areas.

One useful image processing library to use is OpenCV, which contains several advanced contour detection methods, image transformations, and image rotations that can simplify complex x-rays into rectangular boundaries which can be individually focused on for the computation of the Leg Length Discrepancy. To identify these rectangular boundaries, some preliminary transformations must take place: 1) converting to grayscale and 2) applying edge detection.

The process of converting to grayscale is performed, but applying edge detection is a more nuanced process; for example, Laplacian edge detection has many different arguments, from kernel size, ddepth (desired depth), scale, delta, and default border. By analyzing the second derivative of the function, or more specifically, finding zero crossings of the second derivative, the edges can be detected. The Laplacian Operator may be defined as the sum of the pure second derivatives, as indicated below.

Laplacian(f)=d\hat{ }2fdx\hat{ }2+d\hat{ }2fdy\hat{ }2

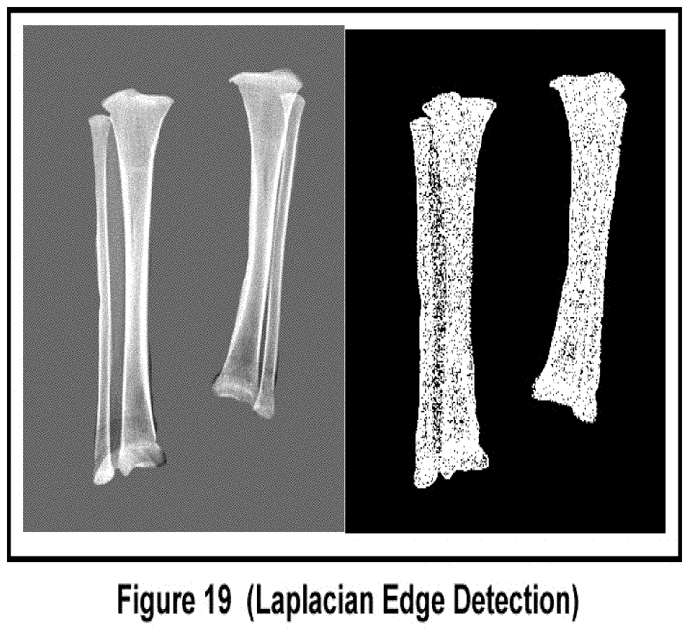

Often, when considering to apply Laplacian edge detections to images, the edge will become lost as there can be infinitely many changes in intensity between neighboring pixels. To combat this, smoothing (such as Gaussian Blur) may be added or applied, before applying Laplacian Edge detections. These preliminary transformations will allow more accurate findings of the two major contours in each image. Reference is made to FIG. 19, which is an example of what a Laplacian Edge Detection may look like on an x-ray of two sets of tibia and fibula; showing on the left side the original image, and further showing on the right side a post Laplacian Edge Detection version which shows the contours/edges more clearly.

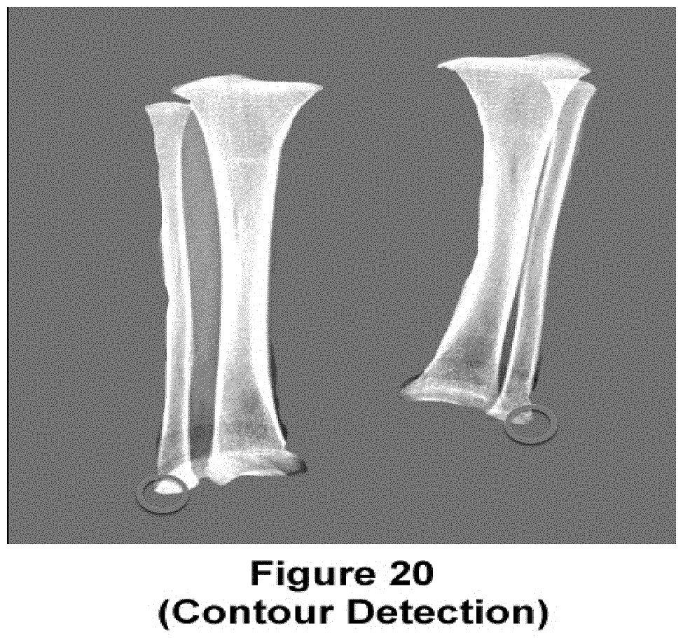

Following these transformations, the major contours must be found. It is difficult to separate the tibia and the fibula in one pass of finding the contours, so first, the general rectangular boundaries that define each set of bones must be identified. Bone contour detection is demonstrated in FIG. 20.

Subsequently, using a contour finder and bounding that contour with a rectangle, each rectangle can then become a new image. These individual images are then appropriately cropped and rotated so that irrelevant minima and maxima can be discarded. For instance, in the image shown in FIG. 20 demonstrating contouring, the minima returned would be the lowest point on the fibula, which should be ignored.

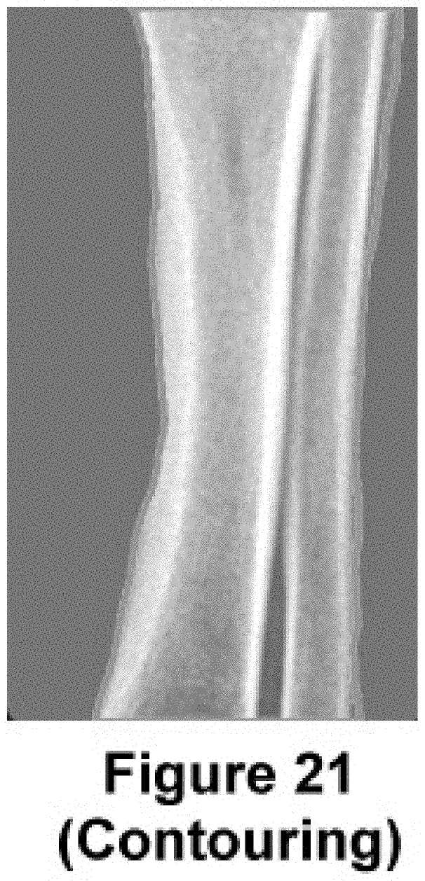

To do this accurately, the vertical middle part of each set of bones can be analyzed. More precisely, if the middle vertical 60% of the bones is considered, the break between the tibia and the fibula can be better identified. The reasoning for this, realized the Applicants, is in the bone structure. These two bones join at the top at the proximal tibiofibular joint. The space between the bones comprises an interosseous membrane, which is much darker than the bones in X-rays. Disregarding the top and bottom of each boneset allows a better analysis, in accordance with some embodiments, because the darker interosseous membrane will be the only separating factor between the tibia and fibula. The darkest column that lies in the boneset will be in this interosseous membrane. Once the image is cropped to 60% of the bone (vertically), two additional steps may be taken for the tibia to be appropriately separated from the fibula: the border should be perfectly straightened so it is upright, and then the darkest portion from the left side of the bone to the right side of the bone should be found (the border itself); as demonstrated in FIG. 21.

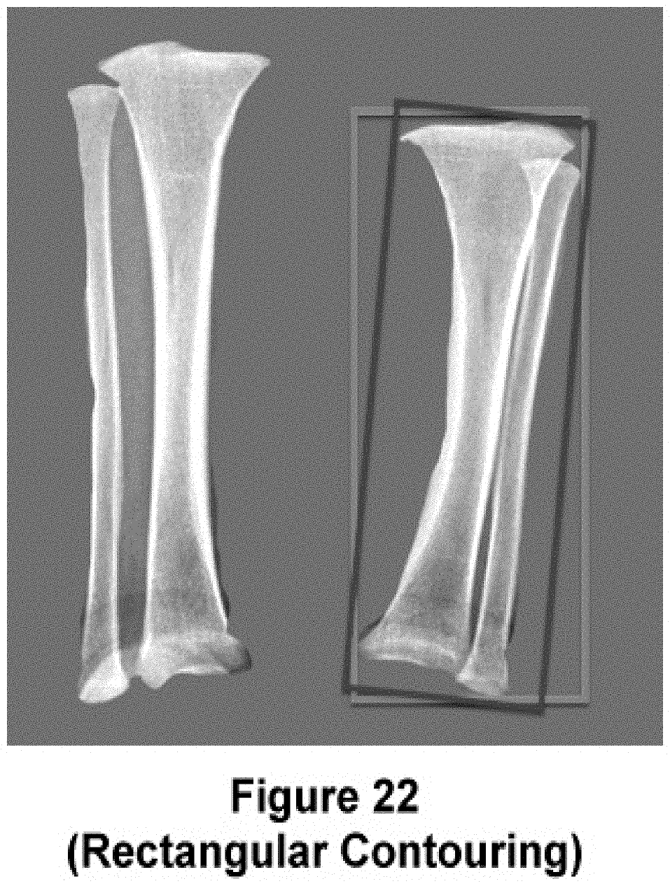

Using the rectangular contour and finding the points that form the rotated rectangular boundary of each boneset, each boneset can then be appropriately rotated so that the boundary between the tibia and fibula is completely upright. An example of using boxPoints( ) vs a non-rotated rectangular boundary is shown in FIG. 22.

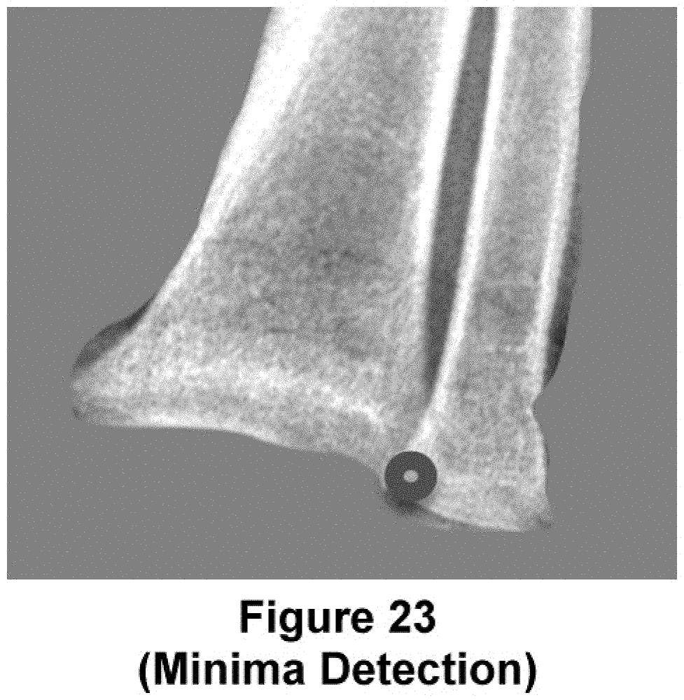

Once making the rotated rectangle upright, finding the border between the tibia and the fibula may be performed by finding the column with the lowest mean. However, the problems that may arise in some situations are, that areas immediately adjacent to the boneset on both sides may be returned as the columns with the lowest mean, so only columns comprising a majority of bone pixels can be considered. There are various ways to achieve this and to overcome such possible problems, in accordance with some embodiments; and one solution is to use the sides of the rectangle bounding the contour of the cropped image (shown in the previous Figures). Alternatively, a fixed portion of the image can be searched, such as only 30-70% of the entire image. Once the column is found in the upright image of the bone, some a math calculation can be done to find the column in the original image that separates the tibia and fibula at the bottom of the bone. The point referred to is shown in FIG. 23, demonstrating such minima detection.

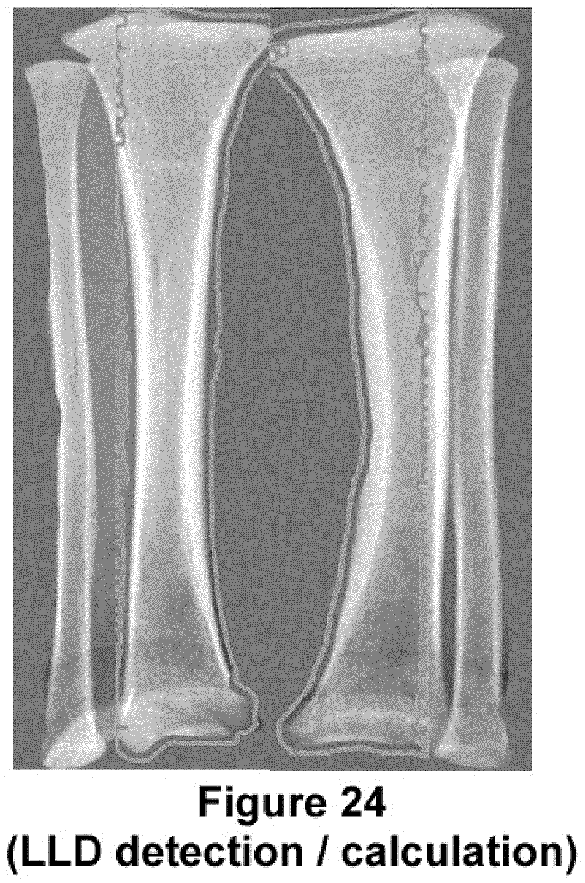

Some embodiments may use one or more methods of finding this point. One method is to use a geometry-based process, to convert from the column in the upright image to the column in the original rotated image. Another method is to rotate each original boneset to the angle that makes it upright (without out any vertical crop). Then, the process continues by finding the minima of each boneset, wherein the start to the column is considered if it is the left boneset, and the column to the end is considered if it is the right boneset. FIG. 24 is an example of the range of possible values that need to be searched in order to find the extreme of each tibia.

Then, using the distance formula, the actual LLD can be determined or computed. All this together forms a calculation of the LLD when the image uploaded is in X-ray form. However, the complexity may increase when the algorithm is identifying and calculating LLD based on a non-X-Ray photograph of a pair of legs. The aim is to provide only a minimum amount of “stenciling”, just so that there is immediate feedback for the user before the image is uploaded, which notifies them that the image passes the initial constraints. The algorithm is configured to identify several points in each leg in order to measure discrepancies between the distances of corresponding points, and ultimately gauge inconsistencies that can point to LLD issues.

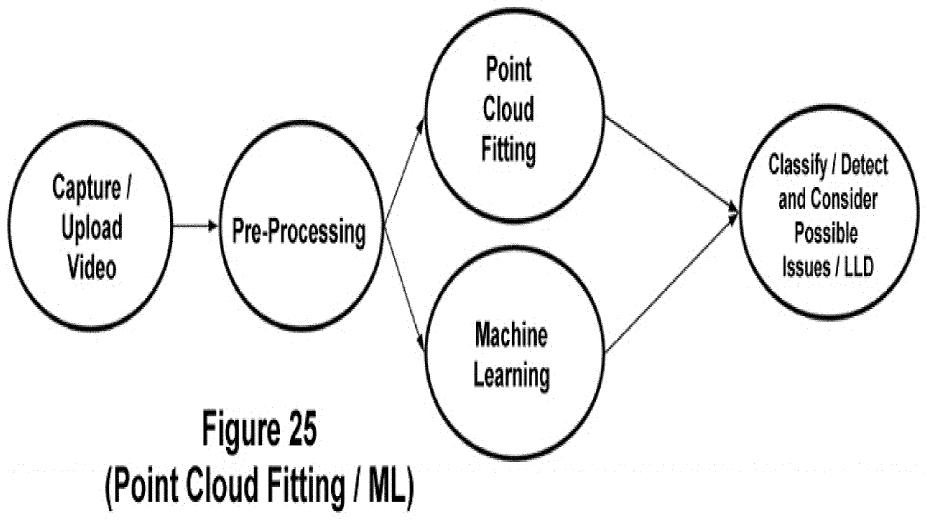

Static recognition of each boneset can be achieved through machine learning, by training an algorithm on marked photos with the joints labeled. In some embodiments, millions of training images may be used to properly train algorithms, but it may increase the complexity when a user seeks to upload a video for a more comprehensive analysis. Using machine learning in conjunction with some “incremental adjusting” techniques (such as point cloud mapping) can prove effective in providing real-time skeletal tracking. The amount with which these are combined can be determined by their purpose. The requirements of diagnostics in apps are high frame rate, and an acceptable image quality, though it is not essential to have the highest quality image to generate a depth map. In this context, FIG. 25 shows a flowchart point fitting via a Machine Learning engine.

The machine learning algorithm may be trained on thousands of annotated images. The approaches towards classification of the leg joints can include convolutional neural networks (CNNs), decision trees, random decision forests, K-nearest neighbors, and Naive Bayes algorithms. Each of these methods will be weighed in their effectiveness in leg tracking, and some will be noted to have greater accuracy by way of their adaptability and scalability.

In some embodiments, Decision Trees can be utilized as a classification problem for each pixel in a frame. The classification problem for a lower body tracker can include identifying the Greater Trochanter, Patella, as well as the joining of the tibia with the talus and navicular bones. The distance between these points can reveal important information about the stance of the user and expose inconsistencies through asymmetry between the left and right bonesets. One decision tree can classify all pixels in an image by a randomizer that estimates depth differences between a parent pixel and randomly picked children pixels.

Conjunction of multiple decision trees can provide more accurate probability distributions of each pixel and what body part it belongs to. Once this is done, utilization of a maximizer can retrieve what body part the algorithm believes the body part is most likely. Rigorously, the lower body classification algorithm fI(x) for an image I may be:

$${f}_{I} ( x ) = [ b : {\sum }_{t ∈ T} ℙ ( {f}_{t} ( x ) = b ) > {\sum }_{t ∈ T} ℙ ( {f}_{t} ( x ) = r ) \& b , r ∈ B \& b \neq r )$$

In this formula:

b is the decided body part classified by the algorithm;

T is the set of all decision trees;

ft(x) is the classification of the pixel according to one decision tree;

r is any other body part;

B is the set of all body parts;

P denotes probability;

For any image I, the classification of a certain pixel will be the body part for which the random forest results in the max sum of the probabilities from each decision tree. Each decision tree will produce a unique probability distribution for each pixel. Summing over the probability that each decision tree believes a pixel to be from a body part will demonstrate the confidence of the algorithm in the classification of a pixel.

System Security Implementation:

The Server and the device may optionally employ Blockchain technology to create distributed databases and/or a self-proving ledger and/or a self-authenticating ledger. The data in the form of images may be stored in the form of a chain or a blockchain that grows incrementally, by appending new images to the end of the current blockchain. This makes each image instance immutable. Each block of data is connected to the previous one with a cryptographic hash function to ensure the integrity and prevent tampering with data. This ensures that the system is robust to attacks, such that even if a single bit in the chain changes, it would require recomputing all hashes from the altered block to the last one, which is a power-consuming task. The system and device combination also leverages the advantage in distribution because it makes the database directly accessible to all the other devices, and is resistant to DDoS attacks against a single central server. This allows the system to be open to all the participating networks, but if there is a rogue device that enters the network or if a hospital network needs to be turned off, while re-allowing the access as needed in a selective manner. This also brings enormous amount of benefits to the network which include shared learning, instantaneous data exchange, automated contract execution, network security, and improved collaboration. This enables ‘Smart Contracts’ type execution for images.

This type of robust security allows this system to be deployed on the Internet in a B2C business model. This can be an Internet of Things type of application, opened for people to subscribe and identify potential conditions related to LLD while gamifying the network and possibly with gamification and to store data and access it with Ethereum smart contracts for doctors or other hospital networks. Since all the data-at-rest (essentially the images after they are used for creating the CNN/DNN algorithms) protecting the integrity of a collection of images is of paramount importance. This is done leveraging ‘Merkle trees’ concept by creating a hash of all files that are organized in a tree, and storing the root of the tree in the blockchain. This configuration provides a very efficient and a suitable way to store the large data structures.

In some embodiments, the system needs to be accessible by multiple people of facilities: For example, image retrieval may happen from multiple data sources; each of the devices in this case may be an IoT device or other type of electronic device. Doctors or experts in hospitals want to improve the accuracy of diagnosis, and can search for similar images over a great number of medical images or common leg images with an already determined diagnosis. The subset of results may form a separate set of images from which a new CNN can be generated, and this would serve as auxiliary diagnosis which overall improves the diagnosis effectiveness.

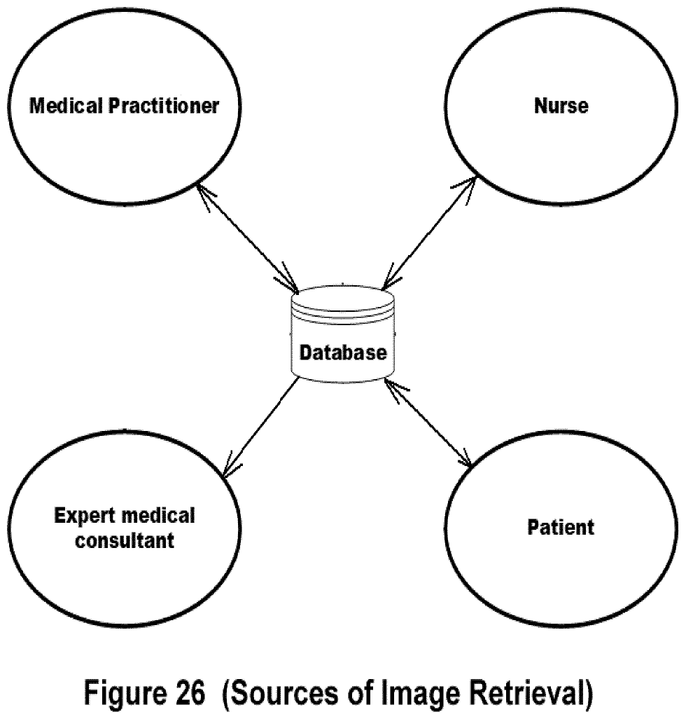

This is illustrated in FIG. 26, which shows sources of data retrieval.

When data exchange like this happens, the key factors to consider are:

(a) Privacy Protection: Prevent revealing personally identifiable information.

(b) Scalability: The number of images can exponentially increase in a B2C type of scenario described. Therefore, the system should be capable of accommodating this exponential growth in terms of the number of participants as well as the size of the images/volume shared.

(c) Reliability: Protect the shared images from being deleted or tampered with by potential attacks. System will also maintain data replicas to provide redundancy.

In the scenario described, image transmission from the database to any of the users will go through a public or private cloud. While there may be end encryption, the interaction information and association relationship of the endpoints may leak, rendering the system vulnerable resulting in the relevant information in the images leaked. Furthermore, an entire server can crash, with a single point of failure. Some embodiments may address these problems by utilizing a blockchain, which is a time-stamped, decentralized series of fixed records, containing data of any size, which is controlled by and accessible by a large network of computers, which are scattered around the globe, and not necessarily owned by any one single organization. Every block is secured and connected with each other block using cryptographic hashing which protects it from being tempered by an unauthorized person.

Since the images (radiological or non-radiological; X-Ray images, or non-X-Ray photographs) are considered electronic health records (EHRs), some embodiments may implement an attribute-based signature scheme with multiple authorities to guarantee the validity of the images. With a de-centralized system, peer-to-peer transactions make it impossible for third parties to steal image privacy information during the retrieval process. Secondly, each node in the network has a copy of the transaction record, which avoids single points of failure. Thirdly, making the transaction information on the blockchain publicly available allows the users to conduct image retrieval over a collection of images shared by either different medical image providers or even by millions of patients worldwide. This scale in images renders higher accuracy while protecting the information from unnecessary privacy disclosure to unintended parties, data tampering by non-essential personnel, and data forgery by random players.

For this application, the block chain structure will consist of the following elements:

(a) A block will always contain a timestamp or data regarding the time when the block was created.

(b) Each block will have a digital finger print which is a unique hash produced by combining all the contents within the block itself.

(c) A block will also always contain a previous hash or a reference to the prior block's hash. This is how blocks chain to one another.

So, the properties of a block in the block chain may be, for example:

(i) Timestamp: The time the block is created determines the location of it on the blockchain.

(ii) Transaction Data: The information to be securely stored in the block.

(iii) Cryptographic Hash: A unique code produced by combining all the contents within the block itself—as a digital fingerprint. A cryptographic hash function is used to here, meaning that the output appears to be random (or pseudo-random) but is actually deterministic, i.e., the same input will always produce the same hash, and the output (hash) is irreversible such that it cannot be used to produce the original input.

(iv) Previous Hash: Each block has a reference to the block prior to its hash. This is what makes the blockchain unique because this link will be broken if a block is tampered with.

The general steps for implementation are:

(a) The image data which is based on attribute-based signatures, is stored in JSON format so that it is easy to read. The image data is stored in a block and the block contains multiple data. As multiple blocks get added, to differentiate one from another fingerprinting is used.

(b) The fingerprinting is implemented by using hash and to be particular, we will use the SHA256 hashing algorithm. Every block will contain its own hash and also the hash of the previous function so that it cannot get tampered.

(c) This fingerprinting will be used to chain the blocks together and thus create a blockchain. Every block will be attached to the previous block having its hash and to the next block by giving its hash.

(d) The mining of the new block is done by successfully finding the answer to the proof of work. To make mining hard, the proof of work must be hard enough to get exploited. It is noted that for demonstrative purposes, a proof-of-work blockchain is discussed; however, some embodiments may be implemented using proof-of-stake technology or other suitable proof-based blockchains.

(e) After mining the block successfully, the block will then be added to the chain.

(f) After mining several blocks, the validity of the chain must be checked in order to prevent tampering with the blockchain. For this a webapp is made (e.g., with Flask) and can be deployed locally or publicly as per the need of customers (e.g. large network like a hospital).

(g) The image features can be extracted from the Machine Learning algorithm, which is then encrypted and recorded in blockchain transaction.

The encryption will be carried out by seeding with two same sized very large prime numbers p and q. The RSA modulus is calculated as n=p*q. The greater the modulus size, the higher is the security level of the RSA system. RSA modulus size is 2,048 bits to 4,096 bits. So, we find numbers that are prime with a satisfactorily high level of probability. Steps involved for this are, for example:

(i) Preselect a random number with the desired bit-size.

(ii) Ensure the chosen number is not divisible by the first few hundred primes (these are pre-generated).

(iii) Apply a certain number of Rabin Miller Primality Test iterations, based on acceptable error rate, to get a number which is probably a prime; Set error probability limit to very low (e.g., ½\hat{ }128).

(iv) If the chosen random value passes all primality tests, it is returned as the n-bit prime number. Otherwise, in the case of test-failure, a new random value is picked and tested for primality. The process is repeated until the desired prime is found.

The parameter p is public to all participants, such as hospitals, third parties, patients, and image retrieval service providers in the system while the others are kept secret in the system.

Some embodiments may utilize one or more Alternate ways of measuring pressure points on the feet, for example as described herein:

Insoles are in use for years and can sometimes be used to adjust structural deformities and reduce the stress in threatened areas that are exposed to pressure beneath the foot. Foot orthoses or insoles have several applications and may be useful in diagnosing cause and effect in some cases. How the insole is created depends on its intended use. Orthoses can be custom made to control the velocity and degree of ankle pronation or to redistribute the plantar pressure etc.

To preserve the aesthetics, a custom shoe insert would be easier for a patient to use while walking/running during the normal course of regular activities while gathering real-time pressure points on the feet. In order to implement this, ultra-thin force measuring sensors can be integrated into a shoe insert, to measure and to analyze force distribution on a patient's foot at different points in the feet. The data is then reported back to the system for further analysis; for example, via a Wi-Fi or Bluetooth transmitter or transceiver, which may be in the insole or in a shoe, and which may be connected to a small battery or power source (which also powers the miniature force-measuring sensors). When this insert or insole (or shoe) is configured to notify family members, the same show insert also serves the purpose of security/call for help in the case of patients who have a severe case of LLD or in the case of elderly patients during the event of a fall or accident or other traumatic event; or for detecting that a person is lying down and non-moving or non-walking for a pre-defined time period. The same insert or insole or sole or shoe can also serve the purpose of tactile feedback via the insert to the wearer, when the system determines that the person has deviated from the normal balance posture. This may help elderly patients or those with severe impairments or with particular disabilities.

This is accomplished, for example, by load cells positioned under the foot to map the foot pressure or the force(s) that are applied onto such load cells or force sensors. This type of arrays with load cells may be force plate arrays, suitable for measurement of ground reaction forces of legs during running or walking. The force plate is composed of a plate base and three beams whose surface and sidewall area are doped, which enable to detect pressure and/or shear force. Force plate arrays are accurate and can provide repeatable measurements of pressure values. The frictional force is resulting from two surfaces sliding against each other. The force depends on the weight of the object and the coefficient of friction μs. The value of μs varies depending on the material, the “wetness” (or dryness) of the surface, and the static or dynamic nature. Friction is calculated by the following formula:

F=μs×m×g

wherein g=9.81 m/s2, and m is the mass in kilograms. In addition, there will be stress and strains. For example, Hooke's Law allows quantification to determine the stress and strain as: σ=F/A, where F is the force acting over the cross-sectional area A and the deformation component is calculated from ε=δ/L0, where δ is the change of strut length, and L0 is the original strut length; and depending on the elasticity of the material used, modulus of elasticity and Hooke's law provide a way to calculate the proportional stress and strain.

An alternate solution is to use pressure sensitive films to measure the contact pressure as well as the cavitation pressure. For example, homogeneous opaque films can be used as substrates or overlay to measure Pressure on soles of human feet and on soles of shoes. Adult humans have a foot pressure of between 2.5 lbs/in² and 3.5 lbs/in².

To produce insoles, a representation of the patient feet's is required. This is usually made through casting or with the help of a foam box or by 3D scanning a representation of the feet. This representation of the feet makes it possible to manufacture individualized insoles. Current state of the art technology is either vacuum forming or Additive manufacturing or subtractive manufacturing with ethylene-vinyl acetate (EVA) combined with cork or plastic. The proposed force plate array can be integrated into a standard insole produced by any one of the methods. The insoles have specialized contours which make the material selection and the lamination of pressure sensitive film extremely difficult to mount on to the insoles. The lamination of this pressure sensitive film uses a specialized process for a special type of fabric. This allows for uniform, aesthetically pleasing appearance to flexible printed circuit materials to be embedded into the insole without diminishing key physical properties such as dielectric strength, tensile strength and dimensional stability. In addition to the aesthetics, the adhesion of the film can also provide functional benefits in applications where accuracy for both continuous and momentary pressure measurements are important. Thickness variations in the overall composition of the material is also very important to ensure that it can be inserted into a variety of shoes with varying level of stiffness of outer sole. In some embodiments, force-measuring sensors or films or components may be integrated into an insole or an insert, or into a sole portion of a shoe, or into a shoe itself.

The “Deep Learning” technology refers to a technology by which computers may think and learn like a human, especially to group or categorize objects and data. Deep-learning methods are representation-learning methods with multiple levels of representation, obtained by composing simple but non-linear modules that each transform the representation at one level into a representation at a higher, slightly more abstract level. The key aspect of deep learning is that these layers of features are not designed by human engineers: they are learned from data using a general-purpose learning procedure as used in this invention.

The deep learning is a machine learning technique which is proposed for overcoming the limitation of “Artificial neural network” algorithm. The Deep learning has two kinds of data categorization approach, i.e., supervised learning and unsupervised learning. In the supervised learning approach, a computer is trained with well-categorized information. Some embodiments may use supervised learning.

Deep learning in some embodiments of this invention uses a pipeline of modules all of which are trainable. This allows for multiple stages in the process of recognizing an object and all of those stages are part of the training for subsequent model generations i.e., representations are hierarchical and trained.

How are mobile pictures/photographs taken (Normal Photographs and determination of the LLD based on them):

Normal/non-Xray images (or photographs) will take upon a predetermined format. For these purposes, a mobile app is created that would help normalize all the new images taken and added to the database in a specific format. The images will consist of three formats:

(1) Frontal image of the legs from above the knees;

(2) Rear view of the legs from above the knees;

(3) Side view of the legs with both knees visible, and particularly,

(3a) With right leg in the front,

(3b) With left leg in the front.

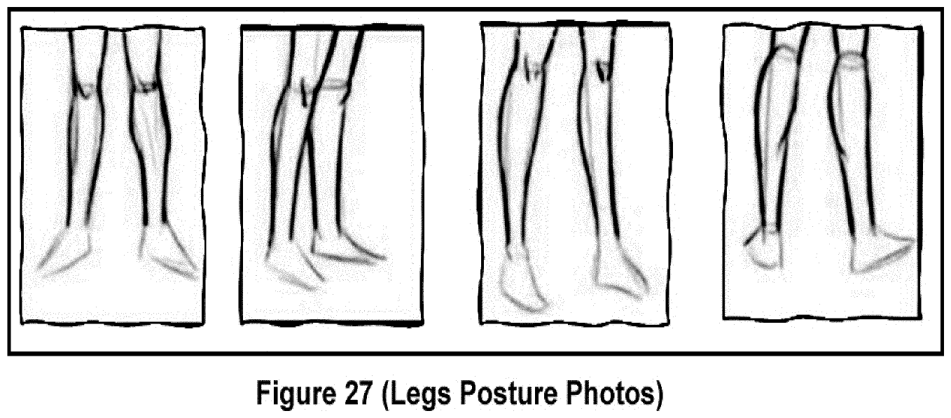

These are depicted in the images of FIG. 27, demonstrating the various leg posture photographs that can be utilized in accordance with some embodiments.

To accomplish the accurate determination of the posture, one or more options may be used, for example:

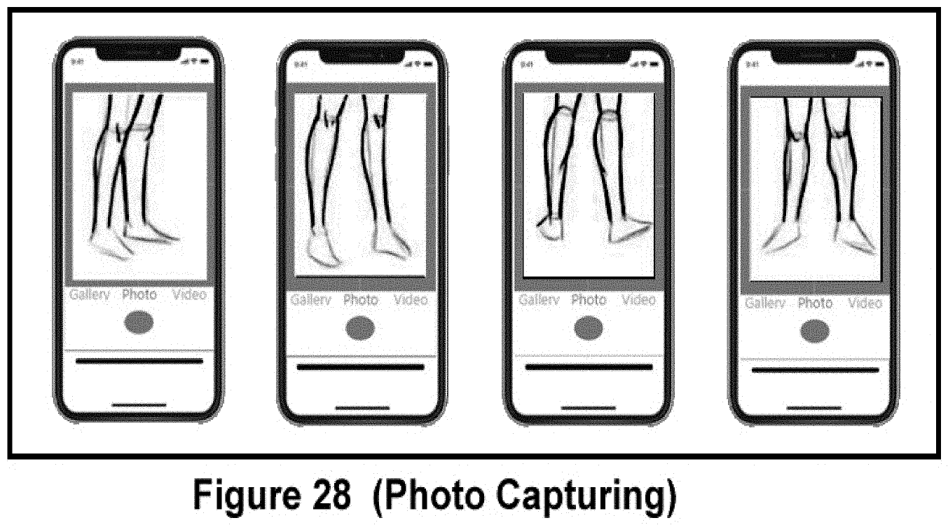

(i) A mobile phone or smartphone based application for capturing images of a particular size (aided by a template) in conjunction with a computer adapted to receive this electronic image file and a photograph generation software component in communication with the computer. The photo capturing is demonstrated in FIG. 28.

(ii) The photograph generation software component is adapted to analyze the electronic image file and present to a user a plurality of image formats from which the user selects a desired format. The method includes the steps of providing an electronic image file to a computer, selecting the electronic image file, and presenting to a user a plurality of image formats from which the user selects a desired format with which to upload the selected electronic image file.

The image is optimized and optionally enhanced for feature extraction as shown and discussed above. Accordingly, some embodiments provide a method and/or system for capturing an image within a template, using a mobile communication device; transmitting the image to a server; and processing the image to create a bi-tonal image for feature extraction. For example, a mobile communication device, such as a camera-equipped phone, or a smartphone or a tablet or other electronic device having a camera and a transceiver (e.g., some smart-watch devices or even some camera-equipped gaming devices) would transmit the image of the legs to the server, where the image is processed and results in a bi-tonal image.

The term “color images” includes, but is not limited to, images having a color depth of 24 bits per a pixel (24 bit/pixel), thereby providing each pixel with one of approximately 16 million possible colors. Each color image is represented by pixels and the dimensions W (width in pixels) and H (height in pixels). An intensity function I maps each pixel in the [W×H] area to its RGB-value. The RGB-value is a triple (R,G,B) that determines the color the pixel represents. Within the triple, each of the R (Red), G (Green) and B (Blue) values are integers between 0 and 255 that determine each respective color's intensity for the pixel.

The term “gray-scale images” includes, but is not limited to, images having a color depth of 8 bits per a pixel (8 bit/pixel), thereby providing each pixel with one of 256 shades of gray. Gray-scale images also include images with color depths of other various bit levels (e.g. 4 bit/pixel or 2 bit/pixel). Each gray-scale image is represented by pixels and the dimensions W (width in pixels) and H (height in pixels). An intensity function I maps each pixel in the [W×H] area onto a range of gray shades. More specifically, each pixel has a value between 0 and 255 which determines that pixel's shade of gray.

Bi-tonal images are similar to gray-scale images in that they are represented by pixels and the dimensions W (width in pixels) and H (height in pixels). However, each pixel within a bi-tonal image has one of two colors: black or white. Accordingly, a bi-tonal image has a color depth of 1 bit per a pixel (1 bit/pixel). The similarity transformation, in some embodiments, utilizes an assumption that there are two images of [W×] and [W′×H′] dimensions, respectively, and that the dimensions are proportional (i.e. W/W′=H/H′). The term “similarity transformation” may refer to a transformation ST from [W×] area onto [W′×H′] area such that the figures should look the same—but yet, one may be of a different size, and/or may be flipped, rotated, or translated relative to the other, and similarity transformation maps pixel p=p(x,y) on pixel p′=p′(x′,y′) with x′=x*W′/W and y=y*H′/H.

Mobile devices that incorporate cameras have also become ubiquitous. The mobile device may include a camera, or might include functionality that allows it to connect to a camera. This connection might be wired or wireless. In this way the mobile device may also connect to an external camera and receive images from the camera; or may have an integrated or built-in camera or imager.

Typically, for systems which utilize images captured on the mobile device, the process of evaluating an image to determine if it is of sufficient image quality can become time consuming and cumbersome for the user of the mobile device. Moreover, these images need to be uniform in look and feel for an effective outcome. Therefore, it would be advantageous to streamline and automate the process of capturing such images with mobile devices and verifying that the quality of the image is sufficient for processing. Such images can vary quite a bit depending on the resolution, picture depth, height etc. For this, some embodiments may ensure uniformity in:

(a) high resolution photo that is not blurry, grainy, or pixelated.

(b) Standard square size (eg m×m inches)—Image pixel dimensions of a square aspect ratio (meaning the height must be equal to the width). Minimally 600 pixels (width)×600 pixels (height) to a maximum of 1200 pixels (width)×1200 pixels (height).

(c) Use a white background.

(d) Use of markers for image processing—a large sheet of white paper to tape up on the wall with the markers affixed on the paper.

(e) Using a flash or illumination unit, or not having the subject stand in direct sunlight, to avoid shadows in the photo.

Mobile Device Automatic Capture of Still Picture or Photograph:

(i) The mobile device is configured to automatically capture an image of the subject when certain parameters are met.

(ii) This involves analysis of various position settings of the mobile device, its internal image sensor and the ambient environment to get the subject in focus. Here, an additional feature may be implemented to automatically capture the picture when the settings are met without the user pressing a button can be done.

(iii) This serves as the first step of capturing of the image to the prior end-to-end solution implementation where images are processed and the algorithm is run to identify the LLD in the subject image. The mobile device application provides the user with tools and information to improve the quality of the image and reduce errors from poor image quality or improper position.

(iv) When a desired higher quality image is uploaded, that reduces the chance that the image will be rejected by the server, which would otherwise require the user to capture another image of the patient's legs. So, moving this functionality to the end device (leg minder) problems with the captured image can be immediately identified and corrected. This will prevent the time and processing associated with image transmission to the server, complex image processing and analysis at the server, and response from the server back to this user.

(v) By utilizing this technology within a mobile capture device helps the user to capture a high-quality image, reduced post-capture image processing.

Mobile Device Automatic Capture of a Video:

(I) The above-mentioned benefits can also be derived by a video-based mobile image capture, such that the user utilizes an electronic device to capture a video, which in turn comprises of series of discrete frames (e.g., 30 frames per second).

(II) While the user takes a photo of a patient's legs, the image capture application will be automatically switched into a video capturing mode. The video frames are immediately captured (via OpenCV API) and are stored in a buffer. As the video frames are captured, selected frames are pre-processed on the mobile device to determine the image's suitability criteria for upload to the server. This allows image quality assessment to evaluate focus, exposure, contrast, shadows, reflection, and other criteria as defined by customized and dynamic settings resident on the mobile device or received from the server. Those frames that do not meet the criteria are quickly discarded, and another frame is then selected for pre-processing. This pre-processing continues until an acceptable frame is found, at which time the video stream is stopped and the user receives a message that the image capture process is complete.

(III) The parameters are measured in real time, to meet certain image quality thresholds.

(IV) These image quality thresholds can be individual parameters being measured, or can be a set of threshold parameters or can be based on a computed total overall quality score. The pass/fail criteria for such thresholds are user defined or are configurable in the server and can be downloaded to each application device.

(V) Over time, the device dependencies are computed and stored on the server for image quality based on the above parameters. This will allow the server to make any auto adjustments depending on the device ID so that the threshold value of one or more parameters can be adjusted based on the computed image quality score.

Template for the Mobile Phone:

A user may be provided with a template in the form of leg and hip outline shape or a quadrilateral on the display screen of the mobile device to guide the user such that the patient's legs are fully contained in the template during the image capture process. The template on the screen may also give the indication to the user to move closer or to move further or move up or to move down. Various user-friendly intuitive icons/colors/vibration/audio cues may be generated for this purpose.

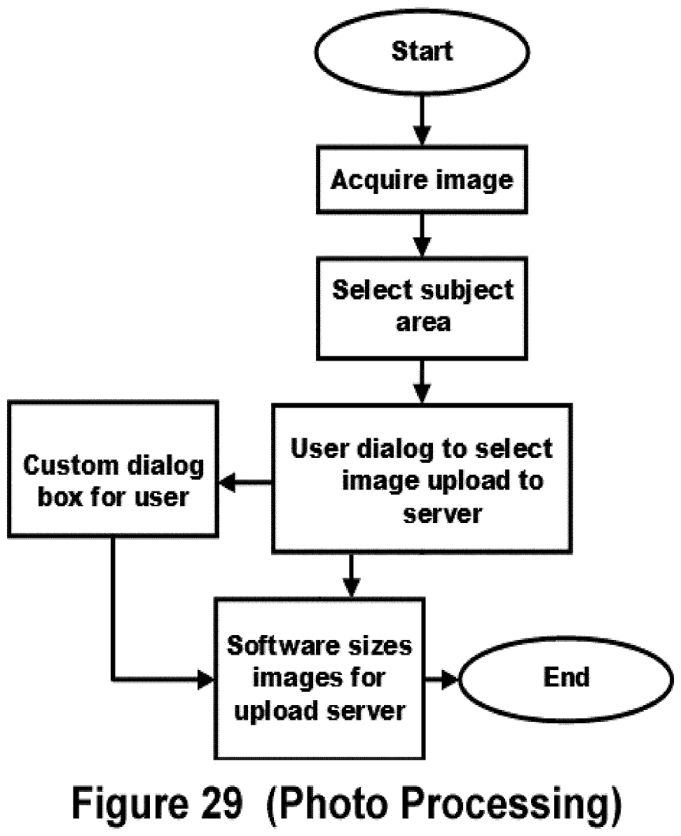

The template aids to fit the desired image size, stability, contour detection and orientation angle. The template may be user specific for example adult male vs female, pediatric male vs female, or may be user selectable based on age group and gender. Since the tilt of the camera is also equally important, the display screen/template will guide the user to change the orientation of the camera to the desired level, to remove any distortion and skew in the images. In some embodiments, the Template may be implemented as a see-through over-layer, or as a partially-gray or partially-opaque layer, or as a border, or as on-screen markers or indicators, or as four-corners on-screen indicators. Additionally, FIG. 29 shows a flowchart of processing or utilizing photographs or non-X-Ray images, in accordance with some embodiments.

Metrics to Measure the “Desired Image” Quality:

A variety of metrics might be used to detect an out-of-focus image. For example, the Brenner function score is a measure of the texture in the image. An in-focus image has a high Brenner function score, and contains texture at a smaller scale than an out-of-focus image. Conversely, an out-of-focus image has a low Brenner function score, and does not contain small-scale texture. Some embodiments may utilize, for ML or NN based analysis of images, only photograph that have a Brenner function score that is greater than a pre-defined threshold value, while discarding other photographs from such training set or validation set or analysis candidates. Alternatively, a focus measure could be employed.

Dynamic Leg Length Measurements:

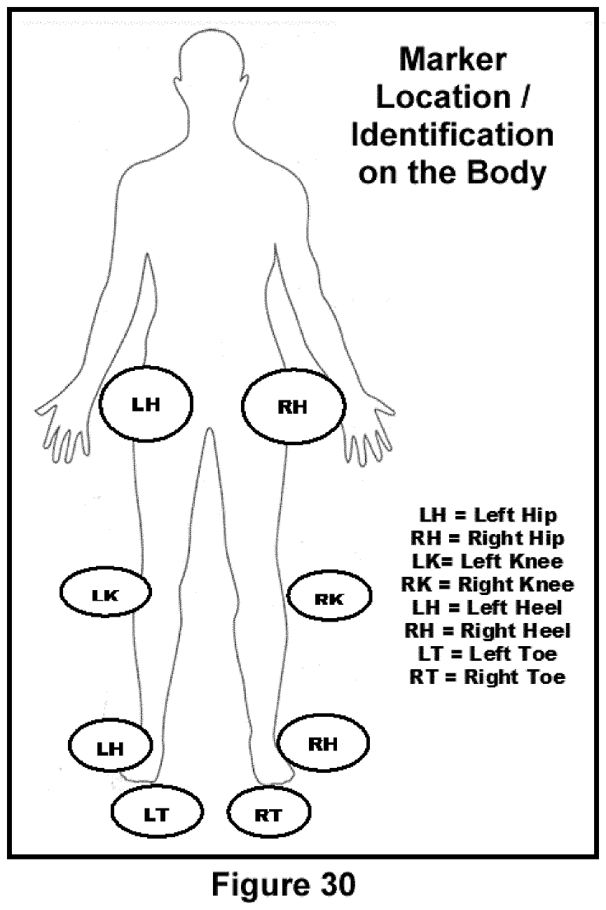

The direct bone length measurement method measures the distance between the anterior superior iliac spine and the medial malleolus. The indirect method measures the differences in leg length between the sides while standing. However, measuring all the components involving leg lengths, for example: joint contractures, static or dynamic mechanical axial malalignment due to structural deformities or muscle weakness or shortening, cannot all be accounted for in this method. To properly measure this, a gait cycle measurement approach May be used. In some embodiments' approach to measure gait, the system may capture movement by video streams captured by cameras, depth cameras and sensors, wearable sensors, and/or recorded videos. Body movement generates tens of thousands of data points a minute while moving through the 3 spatial planes (length, width, and depth). Videos and camera streams are crowded with a lot of other objects in the scene that makes extracting body joint locations a more complex task. Therefore, some systems can use “body landmarks” (e.g., as demonstrated in FIG. 30, showing such body landmarks and their locations) with passive and/or reflective markers per the PlugInGait (PGM) protocol or similar protocol to locate certain points of interest as shown. Such markers appear as a middleware software library (e.g., Wrnch, OpenPose and Azure Kinect body tracker), which is mainly dependent on a pre-trained ML and DL algorithms for skeletal tracking and joints positions extracted from different video frames.

Gait patterns can then be established using a standard laboratory evaluation system that uses a motion analysis system (for example: Vicon, Oxford Metrics, Oxford, UK), following the PlugInGait model (PGM), to measure gait deviations in the lower extremities and pelvis. Patients can walk at their natural/self-selected speed. Thus, the system of some embodiments can automatically perform steering of one or more Narrow Field of View (NFOW) cameras to a target subject and generate un-binned depth mode and outputs at 16:9 resolution of 1080p RGB at 30 fps including inertial measurement units (IMU) data.

The position and orientation of each of the joints can be extracted, for example, as a right-handed joint coordinate system. These form absolute coordinates in the depth camera 3D coordinate system. Thus, in accordance with some embodiments, each joint will be a quaternion represented by q=w+ix+jy+kz=a+v. The system may then normalize quaternions or convert them to unit quaternions by:

Uq=q÷∥q∥

Wherein:

$${U}_{q} = \frac{w}{d} + i \cdot \frac{x}{d} + j \cdot \frac{y}{d} + k \cdot \frac{z}{d}$$

And wherein:

d=∥q∥=\sqrt{w²+x²+y²+z²}

Some embodiments may utilize an Augmented Reality marker, which is is an image that can be recognized by an AR-enabled mobile app and triggers certain augmented reality features. Such markers are typically placed on flat surfaces; as bumpy, irregular or rounded surfaces may deform marker images. A computer vision algorithm will consider all angles to be an additional marker and only be able to pair the AR content with one of the angles. A self-service AR creator is used to combine a marker image with the desired AR content. The user would need to download a mobile app to scan the marker image (depicted in figure X). This approach allows centralized updates of the application and enables dissemination of such apps that are updated centrally. The software then recognizes the image and pairs it with the previously prepared AR content, displaying it on the device's display in real-time.

Some embodiments may utilize an ML model or NN model that was trained on, and/or that is configured or optimized to analyze, images or photographs that are augmented with such AR markers, and/or with Body Landmarks markers or indicators; as such model or engine may have improved results, in certain situations, relative to utilization of photographs that lack such landmarks or markers.

In some embodiments, the ML or NN model is trained on, and/or is utilized for analyzing, only a single frame out of a video clip of multiple frame; wherein the single frame is selected by the system based on a plurality of parameters that are pre-defined as making it the best-available frame for ML or NN purposes. It is noted that in some embodiments, the particular video frame which appears to be the most pleasant to a human observer, is not necessarily the most useful video frame for the purposes of training the ML or NN engine, or for the purposes of feeding such frame into a ML/NN based classifier or prediction unit; but rather, realized the Applicants, a particular video frame which may appear (for example) out of focus and/or having non-pleasant darkness may actually be more beneficial for ML/NN applications. Therefore, some embodiments may define a set of parameters and their respective values; may examine discrete frames of a video clip; and may select a single frame from such video, that has the best set of values of these parameters that are pre-defined as the most advantageous for ML/NN purposes, rather than for human observation purposes.

In some embodiments, the ML/NN engine is trained on, and is validated on, and is then utilized to classify, photographs that are non-X-Ray images; each such photograph showing a pair of legs of a human. In some embodiments, at least some of those photographs (or most of them, or all of them) show the pair or legs from the right side, as a side photograph and as a non-frontal photograph. In some embodiments, at least some of those photographs (or most of them, or all of them) show the pair or legs from the left side, as a side photograph and as a non-frontal photograph. In some embodiments, at least some of those photographs (or most of them, or all of them) show the pair or legs from the right side or from the left side, as a side photograph and as a non-frontal photograph. In some embodiments, at least some of those photographs (or most of them, or all of them) show the pair or legs from an angle, such that the view is non-frontal and non-rear and non-right and non-left, but rather at an angle (e.g., 30 or 40 or 50 degrees).

In some embodiments, the ML/NN engine may detect LLD based on a Binary Type classifier or predictor, which classifies a fresh photograph or a candidate photograph into one of exactly two possible classes which are: (i) a first class, in which the photograph indicates that an LLD condition exists beyond a pre-defined threshold value of certainty; or (ii) a second class, in which the photograph indicates that an LLD condition was not detected (at all, or beyond said pre-defined threshold value of certainty). Accordingly, the ML/NN model may be trained and configured to implement such Binary Type classifier, that classifies a given photograph according to these exact two possible classes.

In some embodiments, the ML/NN engine may detect LLD based on a Ternary Type classifier or predictor, or a Three-Classes classifier or predictor, which classifies a fresh photograph or a candidate photograph into one of exactly three possible classes which are: (I) a first class, in which the photograph indicates that a Severe LLD condition exists (e.g., the engine determines or estimates that there is an LLD condition and that it manifests at least D millimeters of leg length discrepancy, wherein D is a particular pre-defined threshold value, such as 6 millimeters); or (II) a second class, in which the photograph indicates that a Mild LLD condition exists (e.g., the engine determines or estimates that there is an LLD condition and that it manifests not more than D millimeters of leg length discrepancy, wherein D is a particular pre-defined threshold value, such as 6 millimeters); or (iii) a third class, in which the photograph indicates that an LLD condition was not detected (at all, or beyond a pre-defined threshold value of certainty for detection of LLD). This approach may be innovative as it may divert from a conventional approach in some ML/NN systems, that typically classify certain data-points into exactly one out of exactly two possible classes.

Some embodiments may utilize an Over-Fitting Prevention Unit and also an Under-Fitting Prevention Unit; which may perform, respectively, one or more of the above-described techniques in order to prevent (or reduce, or eliminate, or minimize) over-fitting and under-fitting (respectively) of the ML/NN model relative to the data (namely, the photographs or images that are utilized for LLD detection or classification). In some embodiments, the over-fitting prevention operations and/or the under-fitting prevention operations are particularly tailored to the particular domain of LLD detection based on X-Ray images or based on non-X-Ray photographs.

Some embodiments include a computerized method, which is implementable by using at least: one or more hardware processors that are configured to execute code, and that are operably associated with one or more memory units that are configured to store code; wherein the computerized method comprises: determining whether a particular subject has a Leg Length Discrepancy (LLD), by performing: (a1) receiving a training set of images of legs of patients; (a2) receiving a validation set of images of legs of patients; (b) operating on the training set of images by: (b1) performing image normalization and image resizing on said images of legs of patients; (b2) modifying the images of the training set, by applying one or more image transformation operations selected from the group consisting of: image rotation, image flip, skewing, zoom modification, isotropic scaling, shear transformation; (b3) performing a binary-type classification of said images of legs of patients, into exactly one of: (i) a first class of images that includes only images that are determined to not be associated with LLD, or (ii) a second class of images that includes both images that are determined to be associated with LLD and images that are determined to possibly be associated with LLD; (b4) passing the images of the training set of images via convolutions and extracting a first set of unique features from said images of the training set; and operating a Convolutional Neural Network (CNN) unit which applies convolution, kernel initialization, pooling, activation, padding, batch normalization, and stride to the images, to detect one or more particular image-features that are determined to be predictive for LLD detection; (b5) performing pooling and image traversal, through a particular path of convolutions that was passed in step (b4), and concurrently extracting a next set of unique features from said images of the training set by using computerized-vision object detection and computerized-vision pattern recognition; (b6) stacking multiple sets of convolutions that were passed in step (b4), and also stacking multiple pooling layers that were pooled in step (b5), to generate reduced-size images; (b7) feeding the reduced-size images into one or more dense layers of said CNN unit; (b8) applying a SoftMax classifier to reduce binary loss, and further applying a sigmoid classifier; (b9) adjusting a learning rate of said CNN unit for convergence into a solution; (b10) generating by said CNN unit a single-neuron output with a sigmoid activation, which indicates a binary-type output with regard to a particular image; wherein the binary-type output is either (i) the particular image is not associated with LLD, or (ii) the particular image is associated or is possibly associated with LLD; and, (c) operating on the validation set of images by: performing steps (b1) through (b10) on the validation set of images to verify an accuracy of classifications performed by said CNN unit.

In some embodiments, said images of legs of patients include both (i) X-Ray images of legs of patients and (ii) photographic non-X-Ray images of legs of patients.

In some embodiments, said images of legs of patients include, exclusively, X-Ray images of legs of patients.

In some embodiments, said images of legs of patients include, exclusively, photographic non-X-Ray images of legs of patients.

In some embodiments, the method further comprises: collecting said images of legs of patients at a central server, from a plurality of remote imaging devices that are located at a plurality of remote locations; generating a unified Deep Neural Network (DNN) model based on said images of legs of patients that were collected from said plurality of remote imaging devices that are located at said plurality of remote locations; wherein the DNN model is configured to reduce bias or to eliminate bias in diagnosis of LDD by performing training and convolutions on said images of legs of patients that were collected from said plurality of remote imaging devices that are located at said plurality of remote locations, rather than by relying on legs images from a single source or from a single hospital or from a single locality.

In some embodiments, the method further comprises: operating a security module that secures an integrity of said unified Deep Neural Network (DNN) model from malicious attacks, and that blocks malicious attacks to introduce bad data (i) at said central server at a network level, and (ii) at said plurality of imaging devices at an image ingress level.

In some embodiments, the method further comprises: performing a transfer learning process at said central server, on a dynamically-updated dataset of images of legs of patients; periodically generating at said central server an upgraded DNN model; and periodically sending the upgraded DNN model to the plurality of imaging devices.

In some embodiments, said DNN model is configured to detect LLD of a particular person, based on a group photograph that depicts two or more persons standing together.

In some embodiments, said images of legs of patients include, exclusively, side images of legs of patients, and not frontal images of legs of patients.

In some embodiments, said images of legs of patients include both: (i) side images of legs of patients, and (ii) frontal images of legs of patients.

In some embodiments, the CNN model is developed and is dynamically updated at a central server computer based on images of legs that are uploaded to said central computer server from a plurality of end-user devices; wherein a current version of the CNN model is periodically distributed from said central server computer to said end-user devices, and dynamically replaces on said end-user devices a prior version of the CNN model; wherein central upgrading of the CNN model, based on images of legs that are uploaded to said central computer server from a plurality of end-user devices that are located at a plurality of different locations, causes the CNN model and the determining of LLD to be more resilient to bias.

Some embodiments include a computerized system, which is implemented by utilizing at least: one or more processors that are configured to execute code, and that are operably associated with one or more memory units that are configured to execute code. The system comprises: (a) a plurality of distributed end-user devices; wherein each end-user device is an electronic device selected from the group consisting of: a smartphone, a tablet, an electronic device comprising a processor and an imager; wherein each end-user device is configured to acquire digital non-radiological non-X-Ray photographs of legs of persons; wherein each end-user device is configured to perform: (i) a learn-and-predict process, (ii) a Deep Neural Network (DNN) model upgrade process, and (iii) a database transfer process; wherein each end-user device locally-stores therein, and locally-runs therein, a local version of a DNN model that is periodically updated by a central computer server. The system further includes: (b) said central computer server, that is configured to communicate separately, over Internet-based communication links, with each one of the plurality of distributed end-user devices. The central computer server comprises a DNN Engine, that is configured to perform: (i) an initial learning process, (ii) a transfer learning process, (iii) a further database transfer process, and (iv) a further DNN model upgrade process; wherein the DNN Engine periodically upgrades the DNN model, and periodically distributes an upgraded DNN model to each one of said end-user devices. At least one of: (I) an end-user device (out of the plurality of end-user devices), (II) said central computer server, is configured to utilize said upgraded DNN model to generate a determination for LLD diagnosis, indicating whether or not a particular subject has a Leg Length Discrepancy (LLD), by feeding a digital non-radiological non-X-Ray photograph of legs of said particular subject into said upgraded DNN model, based on output from a sigmoid-activated single-neuron of said DNN model. An accuracy of diagnosis of LLD, by each of the plurality of end-user devices or by said central server, gradually improves based on cumulative DNN learning by the central computer server which is based on analysis of images from the plurality of end-user devices.

In some embodiments, the central server computer stores at least: (i) a first version of the DNN model, which is currently being utilized for LLD determination by at least one end-user device; and also, (ii) a second version of the DNN model, which is an upgraded version of the DNN model that is more accurate than the first version of the DNN model, and which is pending for distribution to one or more end-user devices.

In some embodiments, each end-user device periodically replaces, a current-version of the DNN model that is stored locally and is utilized locally in the end-user device, with an upgraded-version of the DNN model that is periodically received over an Internet-based communication link from said central computer server.

In some embodiments, each end-user device is equipped with a security module that is configured to block malicious images from being added to a locally-stored dataset of images and from being copied upstream to said central computer server.

In some embodiments, the central computer server comprises: a Master LLD Database which stores images that are utilized by the central computer server to generate and to update the DNN model for detection of LLD; and a Transferred Learning LLD Database which stores images that were received from a particular end-user device and that were not yet utilized for updating the DNN model. In some embodiments, a DNN Model Updater Unit operates to upgrade or improve the DNN model based on the images in the Transferred Learning LLD Database; and wherein content of the Transferred Learning LLD Database is then added to the Master LLD Database of the central computer server.

In some embodiments, said DNN model is configured to detect LLD of a particular person, based on a group photograph that depicts two or more persons standing together.

In some embodiments, said images of legs of patients include, exclusively, side images of legs of patients, and not frontal images of legs of patients.

In some embodiments, said images of legs of patients include both: (i) side images of legs of patients, and (ii) frontal images of legs of patients.

In some embodiments, the CNN model is developed and is dynamically updated at the central server computer based on images of legs that are uploaded to said central computer server from said plurality of end-user devices; wherein a current version of the CNN model is periodically distributed from said central server computer to said end-user devices, and dynamically replaces on said end-user devices a prior version of the CNN model; wherein central updating of the CNN model, based on images of legs that are uploaded to said central computer server from a plurality of end-user devices that are located at a plurality of different locations, causes the CNN model and the determining of LLD to be more resilient to bias.

Some embodiments provide a computerized system, which is implemented by utilizing at least: one or more processors that are configured to execute code, and that are operably associated with one or more memory units that are configured to execute code. The system is configured to detect Leg Length Discrepancy (LLD) of humans, by applying a Machine Learning algorithm with a Deep Neural Network (DNN) model that classifies digital non-radiological non-X-Ray photographs of legs. The system comprises: (a) a plurality of distributed end-user devices, wherein each end-user device is an electronic device selected from the group consisting of: a smartphone, a tablet, an electronic device comprising a processor and an imager; wherein each end-user device is configured to acquire digital non-radiological non-X-Ray photographs of legs of persons; wherein each end-user device is configured to perform: (i) a learn-and-predict process, (ii) a Deep Neural Network (DNN) model upgrade process, and (iii) a database transfer process; wherein each end-user device locally-stores therein, and locally-runs therein, a local version of a DNN model that is periodically updated by a central computer server; and also, (b) said central computer server, that is configured to communicate separately, over Internet-based communication links, with each one of the plurality of distributed end-user devices. The central computer server comprises a DNN Engine, that is configured: (b1) to receive an initial training set of digital non-radiological non-X-Ray photographs of legs of persons, (b2) to generate from said initial training set an initial DNN model, that is capable of classifying a particular new digital non-radiological non-X-Ray photograph either as manifesting LLD or as non-manifesting LLD, (b3) to receive, from time to time, from a particular end-user device out of said plurality of end-user devices, a copy of additional digital non-radiological non-X-Ray photographs that were captured by said particular end-user device and that were already classified as manifesting LLD or non-manifesting LLD based on a current version of the DNN model that is installed in said particular end-user device, (b4) to add said additional digital non-radiological non-X-Ray photographs to a master database utilized by said central computer server, (b5) to update said initial DNN model based on cumulative DNN learning derived from said additional digital non-radiological non-X-Ray photographs. The DNN Engine periodically upgrades the DNN model, and periodically distributes an upgraded DNN model to each one of said end-user devices. At least one of: (I) an end-user device out of the plurality of end-user devices, (II) said central computer server, is configured to utilize said upgraded DNN model to generate a determination for LLD diagnosis, indicating whether or not a particular subject has a Leg Length Discrepancy (LLD), by feeding a digital non-radiological non-X-Ray photograph of legs of said particular subject into said upgraded DNN model, based on output from a sigmoid-activated single-neuron of said DNN model.

In some embodiments, an accuracy of diagnosis of LLD, by each of the plurality of end-user devices or by said central computer server, gradually improves based on cumulative DNN learning by the central computer server which is based on analysis of images from the plurality of end-user devices.

In some embodiments, the central computer server comprises: a Master LLD Database which stores images that are utilized by the central computer server to generate and to update the DNN model for detection of LLD; and a Transferred Learning LLD Database which stores images that were received from a particular end-user device and that were not yet utilized for updating the DNN model; wherein a DNN Model Updater Unit operates to upgrade and improve the DNN model based on the images in the Transferred Learning LLD Database; and wherein content of the Transferred Learning LLD Database is then added to the Master LLD Database of the central computer server.

In some embodiments, the central server computer stores at least: (i) a first version of the DNN model, which is currently being utilized for LLD determination by at least one end-user device; and also, (ii) a second version of the DNN model, which is an upgraded version of the DNN model that is more accurate than the first version of the DNN model, and which is pending for distribution to one or more end-user devices.

In some embodiments, each end-user device periodically replaces, (I) a current-version of the DNN model that is stored locally and is utilized locally in the end-user device, with (II) an upgraded-version of the DNN model that is periodically received over an Internet-based communication link from said central computer server.

In some embodiments, each end-user device is equipped with a security module that is configured to block malicious images from being added to a locally-stored dataset of images and from being copied upstream to said central computer server.

In some embodiments, said DNN model is configured to detect LLD of a particular person, based on a group photograph that depicts two or more persons standing together; wherein said DNN model is trained on pre-classified group photographs, wherein each of said pre-classified group photographs depicts two or more persons standing together; wherein each of said pre-classified group photographs is classified into exactly one of exactly two classes that are: (i) a first class, in which the group photograph manifests LLD of at least one depicted person, and (ii) a second class, in which the group photograph does not manifest LLD of any depicted person.

In some embodiments, said images of legs of patients include, exclusively, side images of legs of patients, and not frontal images of legs of patients; wherein said DNN model is trained on a pre-classified set of images, that depict side-views of legs of patients, and that are pre-classified as either manifesting LLD or non-manifesting LLD.

In some embodiments, said images of legs of patients include, exclusively, at-an-angle images of legs of patients, which are non-frontal images and are non-rear-side images and non-right-side images and are non-left-side images of legs; wherein said DNN model is trained on a pre-classified set of images, that depict at-an-angle images of legs of patients, and that are pre-classified as either manifesting LLD or non-manifesting LLD.

In some embodiments, said images of legs of patients include both: (i) side images of legs of patients, and (ii) frontal images of legs of patients; wherein said DNN model is trained on both (I) a first pre-classified set of images that depict side-views of legs of patients and that are pre-classified as either manifesting LLD or non-manifesting LLD, and also (II) a second pre-classified set of images that depict front-views of legs of patients and that are pre-classified as either manifesting LLD or non-manifesting LLD.

In some embodiments, the DNN model is developed and is dynamically updated at the central computer server based on images of legs that are uploaded to said central computer server from said plurality of end-user devices; wherein a current version of the DNN model is periodically distributed from said central server computer to said end-user devices, and dynamically replaces on said end-user devices a prior version of the DNN model; wherein central updating of the DNN model, based on images of legs that are uploaded to said central computer server from a plurality of end-user devices that are located at a plurality of different locations, causes the DNN model and the determining of LLD to be more resilient to bias; wherein the DNN model is configured to reduce bias or to eliminate bias in diagnosis of LDD by performing training and convolutions on said images of legs of patients that were collected from said plurality of remote imaging devices that are located at a plurality of remote locations, rather than by relying on legs images from a single source or from a single hospital or from a single geographical region.

In some embodiments, a first end-user device of said plurality of end-user devices, is located in a first geographical region and is operated by a first operator, and thus suffers from a first level of bias; wherein a second end-user device of said plurality of end-user devices, is located in a second, different, geographical region and is operated by a second, different operator, and thus suffers from a second level of bias; wherein the DNN model is dynamically updated at the central computer server based on images of legs that are uploaded to said central computer server from said plurality of end-user devices that comprise said first end-user device having said first level of bias and said second end-user device having said second level of bias; wherein central updating of the DNN model, based on images of legs that are uploaded to said central computer server from the plurality of end-user devices that are located at a plurality of different locations and are operated by a plurality of different operators, causes the DNN model and detection of LLD to be more resilient to bias.

In some embodiments, at least one of the end-user devices is configured to capture a video clip that depicts legs of a person, and is further configured to select only a single particular video frame from said video clip; wherein only said single particular video frame, and not other video frames of said video clip, is used for local in-device LLD detection; wherein only said single particular video frame, and not other video frames of said video clip, is uploaded from said end-user device to a master LLD database of said central computer server; wherein said single particular video frame is selected, locally within said end-user device, not based on its being a video frame having highest visible qualities to a human observer, but rather, based on being a video frame having the highest values of parameters that indicate image suitability for classification by a DNN-based classifier that classifies images based on manifestation or non-manifestation of LLD.

In some embodiments, at least some of the digital non-radiological non-X-Ray photographs, that are used for training the DNN model, include Body Landmarks indicators that are placed on particular body-parts or body-locations of humans that are depicted in said photographs; wherein the DNN model is trained on a training set of images that include digital non-radiological non-X-Ray photographs that show include Body Landmarks indicators.

In some embodiments, at least some of the digital non-radiological non-X-Ray photographs, that are used for training the DNN model, include Augmented Reality Markers that are placed on particular body-parts or body-locations of humans that are depicted in said photographs; wherein the DNN model is trained on a training set of images that include digital non-radiological non-X-Ray photographs that show include Augmented Reality Markers.

In some embodiments, said digital non-radiological non-X-Ray photographs, that are used for training the DNN model and/or for LLD classification, are stored in a blockchain that prevents content tampering.

In some embodiments, said digital non-radiological non-X-Ray photographs, that are used for training the DNN model and/or for LLD classification, are stored in a blockchain that prevents content tampering.

In some embodiments, the central computer server further comprises: (A) an Over-Fitting Prevention Unit, that is configured to prevent or reduced over-fitting of the DNN model to digital non-radiological non-X-Ray photographs, (A1) by performing one or more randomly-selected transformations to existing images of pairs of legs, and creating a set of image variants which is used for increasing a diversity and a total number of training examples of pairs-of-legs, and also (A2) by removing one or more randomly-selected images from said existing images of pairs of legs during a training gradient or a training iteration; and also, (B) an Under-Fitting Prevention Unit, that is configured to prevent or reduce under-fitting of the DNN model relative to digital non-radiological non-X-Ray photographs, by performing at least one of: (B1) adding a hidden layer to the DNN model, (B2) modifying regularization parameters to the DNN model.

In some embodiments, the central computer server is configured to perform a process comprising determining whether a particular subject has a Leg Length Discrepancy (LLD), by performing: (a1) receiving a training set of images of legs of patients; (a2) receiving a validation set of images of legs of patients; (b) operating on the training set of images by: (b1) performing image normalization and image resizing on said images of legs of patients; (b2) modifying the images of the training set, by applying one or more image transformation operations selected from the group consisting of: image rotation, image flip, skewing, zoom modification, isotropic scaling, shear transformation; (b3) performing a binary-type classification of said images of legs of patients, into exactly one of: (i) a first class of images that includes only images that are determined to not be associated with LLD, or (ii) a second class of images that includes both images that are determined to be associated with LLD and images that are determined to possibly be associated with LLD; (b4) passing the images of the training set of images via convolutions and extracting a first set of unique features from said images of the training set; and operating a Convolutional Neural Network (CNN) unit which applies convolution, kernel initialization, pooling, activation, padding, batch normalization, and stride to the images, to detect one or more particular image-features that are determined to be predictive for LLD detection; (b5) perform pooling and image traversal, through a particular path of convolutions that was passed in step (b4), and concurrently extracting a next set of unique features from said images of the training set by using computerized-vision object detection and computerized-vision pattern recognition; (b6) stacking multiple sets of convolutions that were passed in step (b4), and also stacking multiple pooling layers that were pooled in step (b5), to generate reduced-size images; (b7) feeding the reduced-size images into one or more dense layers of said CNN unit; (b8) applying a SoftMax classifier to reduce binary loss, and further applying a sigmoid classifier; (b9) adjusting a learning rate of said CNN unit for convergence into a solution; (b10) generating by said CNN unit a single-neuron output with a sigmoid activation, which indicates a binary-type output with regard to a particular image; wherein the binary-type output is either (i) the particular image is not associated with LLD, or (ii) the particular image is associated or is possibly associated with LLD; (c) operating on the validation set of images by: performing steps (b1) through (b10) on the validation set of images to verify an accuracy of classifications performed by said CNN unit; (d) performing a transfer learning process at said central server, on a dynamically-updated dataset of images of legs of patients; periodically generating at said central server an upgraded DNN model; and periodically sending the upgraded DNN model to the plurality of imaging devices.

Some embodiments provide a a computerized method, which is implemented by utilizing at least: one or more processors that are configured to execute code, and that are operably associated with one or more memory units that are configured to execute code. The method comprises: detecting Leg Length Discrepancy (LLD) of humans, by applying a Machine Learning algorithm with a Deep Neural Network (DNN) model that classifies digital non-radiological non-X-Ray photographs of legs; wherein the method comprises: (a) providing a plurality of distributed end-user devices, wherein each end-user device is an electronic device selected from the group consisting of: a smartphone, a tablet, an electronic device comprising a processor and an imager; wherein each end-user device is configured to acquire digital non-radiological non-X-Ray photographs of legs of persons; wherein each end-user device is configured to perform: (i) a learn-and-predict process, (ii) a Deep Neural Network (DNN) model upgrade process, and (iii) a database transfer process; wherein each end-user device locally-stores therein, and locally-runs therein, a local version of a DNN model that is periodically updated by a central computer server; (b) providing said central computer server, that is configured to communicate separately, over Internet-based communication links, with each one of the plurality of distributed end-user devices; wherein the central computer server comprises a DNN Engine; wherein the method comprises configuring or operating said DNN Engine (b1) to receive an initial training set of digital non-radiological non-X-Ray photographs of legs of persons, (b2) to generate from said initial training set an initial DNN model, that is capable of classifying a particular new digital non-radiological non-X-Ray photograph either as manifesting LLD or as non-manifesting LLD, (b3) to receive, from time to time, from a particular end-user device out of said plurality of end-user devices, a copy of additional digital non-radiological non-X-Ray photographs that were captured by said particular end-user device and that were already classified as manifesting LLD or non-manifesting LLD based on a current version of the DNN model that is installed in said particular end-user device, (b4) to add said additional digital non-radiological non-X-Ray photographs to a master database utilized by said central computer server, (b5) to update said initial DNN model based on cumulative DNN learning derived from said additional digital non-radiological non-X-Ray photographs; wherein the method further comprises: at said DNN Engine, periodically upgrading the DNN model, and periodically distributing an upgraded DNN model to each one of said end-user devices; wherein at least one of: (I) an end-user device out of the plurality of end-user devices, (II) said central computer server, is configured to utilize said upgraded DNN model to generate a determination for LLD diagnosis, indicating whether or not a particular subject has a Leg Length Discrepancy (LLD), by feeding a digital non-radiological non-X-Ray photograph of legs of said particular subject into said upgraded DNN model, based on output from a sigmoid-activated single-neuron of said DNN model.

Some embodiments provide systems, devices, and methods of determining Anisomelia or Leg Length Discrepancy (LLD) of a subject, by using image analysis and machine learning. A system includes a plurality of end-user devices; each device includes a camera to capture digital non-radiological non-X-Ray photographs of legs of a person; each device further includes a local Deep Neural Network (DNN) engine to perform local classification of images as either manifesting LLD or non-manifesting LLD. The digital non-radiological non-X-Ray photographs are also uploaded from the end-user devices to a central server, which updates and upgrades the DNN model based on transfer learning, and periodically distributes the upgraded DNN model downstream to the end-user devices.

Although portions of the discussion herein relate, for demonstrative purposes, to wired links and/or wired communications, some embodiments are not limited in this regard, but rather, may utilize wired communication and/or wireless communication; may include one or more wired and/or wireless links; may utilize one or more components of wired communication and/or wireless communication; and/or may utilize one or more methods or protocols or standards of wireless communication.

Any one or more of the components or units described above and/or herein may be implemented by using a suitable combination of hardware components and/or software components; for example, a processor, a processing core, a Central Processing Unit (CPU), a Graphics Processing Unit (GPU), a Digital Signal Processor (DSP), a controller, an Integrated Circuit (IC), a logic circuit; a short-term memory unit (e.g., Random Access Memory (RAM), Flash memory); a long-term storage unit (e.g., hard disk drive, solid state drive, Flash drive); an input unit (e.g., keyboard, keypad, touch-screen, mouse, pointing device, microphone); an output unit (e.g., screen, touch-screen, display unit, audio speakers); wired transceivers and/or wireless transceivers (e.g., Ethernet card, Network Interface Card (NIC), modem, Wi-Fi transceiver, cellular transceiver, Bluetooth transceiver); a power source (battery, rechargeable battery, power cell, mains electricity); an Operating System (OS), drivers, applications, and/or other suitable components.

Some embodiments may be implemented by using a special-purpose machine or a specific-purpose device that is not a generic computer, or by using a non-generic computer or a non-general computer or machine. Such system or device may utilize or may comprise one or more components or units or modules that are not part of a “generic computer” and that are not part of a “general purpose computer”, for example, cellular transceivers, cellular transmitter, cellular receiver, GPS unit, location-determining unit, accelerometer(s), gyroscope(s), device-orientation detectors or sensors, device-positioning detectors or sensors, or the like.

Some embodiments may be implemented as, or by utilizing, an automated method or automated process, or a machine-implemented method or process, or as a semi-automated or partially-automated method or process, or as a set of steps or operations which may be executed or performed by a computer or machine or system or other device.

Some embodiments may be implemented by using code or program code or machine-readable instructions or machine-readable code, which may be stored on a non-transitory storage medium or non-transitory storage article (e.g., a CD-ROM, a DVD-ROM, a physical memory unit, a physical storage unit), such that the program or code or instructions, when executed by a processor or a machine or a computer, cause such processor or machine or computer to perform a method or process as described herein. Such code or instructions may be or may comprise, for example, one or more of: software, a software module, an application, a program, a subroutine, instructions, an instruction set, computing code, words, values, symbols, strings, variables, source code, compiled code, interpreted code, executable code, static code, dynamic code; including (but not limited to) code or instructions in high-level programming language, low-level programming language, object-oriented programming language, visual programming language, compiled programming language, interpreted programming language, C, C++, C#, Java, JavaScript, SQL, Ruby on Rails, Go, Cobol, Fortran, ActionScript, AJAX, XML, JSON, Lisp, Eiffel, Verilog, Hardware Description Language (HDL), BASIC, Visual BASIC, MATLAB, Pascal, HTML, HTML5, CSS, Perl, Python, PHP, machine language, machine code, assembly language, or the like.

Discussions herein utilizing terms such as, for example, “processing”, “computing”, “calculating”, “determining”, “establishing”, “analyzing”, “checking”, “detecting”, “measuring”, or the like, may refer to operation(s) and/or process(es) of a processor, a computer, a computing platform, a computing system, or other electronic device or computing device, that may automatically and/or autonomously manipulate and/or transform data represented as physical (e.g., electronic) quantities within registers and/or accumulators and/or memory units and/or storage units into other data or that may perform other suitable operations.

The terms “plurality” and “a plurality”, as used herein, include, for example, “multiple” or “two or more”. For example, “a plurality of items” includes two or more items.

References to “one embodiment”, “an embodiment”, “demonstrative embodiment”, “various embodiments”, “some embodiments”, and/or similar terms, may indicate that the embodiment(s) so described may optionally include a particular feature, structure, or characteristic, but not every embodiment necessarily includes the particular feature, structure, or characteristic. Furthermore, repeated use of the phrase “in one embodiment” does not necessarily refer to the same embodiment, although it may. Similarly, repeated use of the phrase “in some embodiments” does not necessarily refer to the same set or group of embodiments, although it may.

As used herein, and unless otherwise specified, the utilization of ordinal adjectives such as “first”, “second”, “third”, “fourth”, and so forth, to describe an item or an object, merely indicates that different instances of such like items or objects are being referred to; and does not intend to imply as if the items or objects so described must be in a particular given sequence, either temporally, spatially, in ranking, or in any other ordering manner.

Some embodiments may be used in, or in conjunction with, various devices and systems, for example, a Personal Computer (PC), a desktop computer, a mobile computer, a laptop computer, a notebook computer, a tablet computer, a server computer, a handheld computer, a handheld device, a Personal Digital Assistant (PDA) device, a handheld PDA device, a tablet, an on-board device, an off-board device, a hybrid device, a vehicular device, a non-vehicular device, a mobile or portable device, a consumer device, a non-mobile or non-portable device, an appliance, a wireless communication station, a wireless communication device, a wireless Access Point (AP), a wired or wireless router or gateway or switch or hub, a wired or wireless modem, a video device, an audio device, an audio-video (A/V) device, a wired or wireless network, a wireless area network, a Wireless Video Area Network (WVAN), a Local Area Network (LAN), a Wireless LAN (WLAN), a Personal Area Network (PAN), a Wireless PAN (WPAN), or the like.

Some embodiments may be used in conjunction with one way and/or two-way radio communication systems, cellular radio-telephone communication systems, a mobile phone, a cellular telephone, a wireless telephone, a Personal Communication Systems (PCS) device, a PDA or handheld device which incorporates wireless communication capabilities, a mobile or portable Global Positioning System (GPS) device, a device which incorporates a GPS receiver or transceiver or chip, a device which incorporates an RFID element or chip, a Multiple Input Multiple Output (MIMO) transceiver or device, a Single Input Multiple Output (SIMO) transceiver or device, a Multiple Input Single Output (MISO) transceiver or device, a device having one or more internal antennas and/or external antennas, Digital Video Broadcast (DVB) devices or systems, multi-standard radio devices or systems, a wired or wireless handheld device, e.g., a Smartphone, a Wireless Application Protocol (WAP) device, or the like.

Some embodiments may comprise, or may be implemented by using, an “app” or application which may be downloaded or obtained from an “app store” or “applications store”, for free or for a fee, or which may be pre-installed on a computing device or electronic device, or which may be otherwise transported to and/or installed on such computing device or electronic device.

Functions, operations, components and/or features described herein with reference to one or more embodiments of the present invention, may be combined with, or may be utilized in combination with, one or more other functions, operations, components and/or features described herein with reference to one or more other embodiments of the present invention. The present invention may thus comprise any possible or suitable combinations, re-arrangements, assembly, re-assembly, or other utilization of some or all of the modules or functions or components that are described herein, even if they are discussed in different locations or different chapters of the above discussion, or even if they are shown across different drawings or multiple drawings.

While certain features of some demonstrative embodiments of the present invention have been illustrated and described herein, various modifications, substitutions, changes, and equivalents may occur to those skilled in the art. Accordingly, the claims are intended to cover all such modifications, substitutions, changes, and equivalents.

What is claimed is:

1. A computerized system,
which is implemented by utilizing at least: one or more processors that are configured to execute code, and that are operably associated with one or more memory units that are configured to execute code;
wherein the system is configured to detect Leg Length Discrepancy (LLD) of humans, by applying a Machine Learning algorithm with a Deep Neural Network (DNN) model that classifies digital non-radiological non-X-Ray photographs of legs;
wherein the system comprises:
(a) a plurality of distributed end-user devices,
wherein each end-user device is an electronic device selected from the group consisting of: a smartphone, a tablet, an electronic device comprising a processor and an imager;
wherein each end-user device is configured to acquire digital non-radiological non-X-Ray photographs of legs of persons;
wherein each end-user device is configured to perform: (i) a learn-and-predict process, (ii) a Deep Neural Network (DNN) model upgrade process, and (iii) a database transfer process;
wherein each end-user device locally-stores therein, and locally-runs therein, a local version of a DNN model that is periodically updated by a central computer server;
(b) said central computer server, that is configured to communicate separately, over Internet-based communication links, with each one of the plurality of distributed end-user devices;
wherein the central computer server comprises a DNN Engine, that is configured
(b1) to receive an initial training set of digital non-radiological non-X-Ray photographs of legs of persons,
(b2) to generate from said initial training set an initial DNN model, that is capable of classifying a particular new digital non-radiological non-X-Ray photograph either as manifesting LLD or as non-manifesting LLD,
(b3) to receive, from time to time, from a particular end-user device out of said plurality of end-user devices, a copy of additional digital non-radiological non-X-Ray photographs that were captured by said particular end-user device and that were already classified as manifesting LLD or non-manifesting LLD based on a current version of the DNN model that is installed in said particular end-user device,
(b4) to add said additional digital non-radiological non-X-Ray photographs to a master database utilized by said central computer server,
(b5) to update said initial DNN model based on cumulative DNN learning derived from said additional digital non-radiological non-X-Ray photographs;
wherein the DNN Engine periodically upgrades the DNN model, and periodically distributes an upgraded DNN model to each one of said end-user devices;
wherein at least one of: (I) an end-user device out of the plurality of end-user devices, (II) said central computer server, is configured to utilize said upgraded DNN model to generate a determination for LLD diagnosis, indicating whether or not a particular subject has a Leg Length Discrepancy (LLD), by feeding a digital non-radiological non-X-Ray photograph of legs of said particular subject into said upgraded DNN model, based on output from a sigmoid-activated single-neuron of said DNN model.

2. The computerized system of claim 1,
wherein an accuracy of diagnosis of LLD, by each of the plurality of end-user devices or by said central computer server, gradually improves based on cumulative DNN learning by the central computer server which is based on analysis of images from the plurality of end-user devices.

3. The computerized system of claim 2,
wherein the central computer server comprises:
a Master LLD Database which stores images that are utilized by the central computer server to generate and to update the DNN model for detection of LLD; and
a Transferred Learning LLD Database which stores images that were received from a particular end-user device and that were not yet utilized for updating the DNN model;
wherein a DNN Model Updater Unit operates to upgrade and improve the DNN model based on the images in the Transferred Learning LLD Database; and wherein content of the Transferred Learning LLD Database is then added to the Master LLD Database of the central computer server.

4. The computerized system of claim 3,
wherein the central server computer stores at least: (i) a first version of the DNN model, which is currently being utilized for LLD determination by at least one end-user device; and also, (ii) a second version of the DNN model, which is an upgraded version of the DNN model that is more accurate than the first version of the DNN model, and which is pending for distribution to one or more end-user devices.

5. The computerized system of claim 4,
wherein each end-user device periodically replaces, (I) a current-version of the DNN model that is stored locally and is utilized locally in the end-user device, with (II) an upgraded-version of the DNN model that is periodically received over an Internet-based communication link from said central computer server.

6. The computerized system of claim 1,
wherein each end-user device is equipped with a security module that is configured to block malicious images from being added to a locally-stored dataset of images and from being copied upstream to said central computer server.

7. The computerized system of claim 1,
wherein said DNN model is configured to detect LLD of a particular person, based on a group photograph that depicts two or more persons standing together;
wherein said DNN model is trained on pre-classified group photographs, wherein each of said pre-classified group photographs depicts two or more persons standing together; wherein each of said pre-classified group photographs is classified into exactly one of exactly two classes that are: (i) a first class, in which the group photograph manifests LLD of at least one depicted person, and (ii) a second class, in which the group photograph does not manifest LLD of any depicted person.

8. The computerized system of claim 1,
wherein said images of legs of patients include, exclusively, side images of legs of patients, and not frontal images of legs of patients;
wherein said DNN model is trained on a pre-classified set of images, that depict side-views of legs of patients, and that are pre-classified as either manifesting LLD or non-manifesting LLD.

9. The computerized system of claim 1,
wherein said images of legs of patients include, exclusively, at-an-angle images of legs of patients, which are non-frontal images and are non-rear-side images and non-right-side images and are non-left-side images of legs;
wherein said DNN model is trained on a pre-classified set of images, that depict at-an-angle images of legs of patients, and that are pre-classified as either manifesting LLD or non-manifesting LLD.

10. The computerized system of claim 1,
wherein said images of legs of patients include both: (i) side images of legs of patients, and (ii) frontal images of legs of patients;
wherein said DNN model is trained on both
(I) a first pre-classified set of images that depict side-views of legs of patients and that are pre-classified as either manifesting LLD or non-manifesting LLD, and also
(II) a second pre-classified set of images that depict front-views of legs of patients and that are pre-classified as either manifesting LLD or non-manifesting LLD.

11. The computerized system of claim 1,
wherein the DNN model is developed and is dynamically updated at the central computer server based on images of legs that are uploaded to said central computer server from said plurality of end-user devices;
wherein a current version of the DNN model is periodically distributed from said central server computer to said end-user devices, and dynamically replaces on said end-user devices a prior version of the DNN model;
wherein central updating of the DNN model, based on images of legs that are uploaded to said central computer server from a plurality of end-user devices that are located at a plurality of different locations, causes the DNN model and the determining of LLD to be more resilient to bias;
wherein the DNN model is configured to reduce bias or to eliminate bias in diagnosis of LDD by performing training and convolutions on said images of legs of patients that were collected from said plurality of remote imaging devices that are located at a plurality of remote locations, rather than by relying on legs images from a single source or from a single hospital or from a single geographical region.

12. The computerized system of claim 1,
wherein a first end-user device of said plurality of end-user devices, is located in a first geographical region and is operated by a first operator, and thus suffers from a first level of bias;
wherein a second end-user device of said plurality of end-user devices, is located in a second, different, geographical region and is operated by a second, different operator, and thus suffers from a second level of bias;
wherein the DNN model is dynamically updated at the central computer server based on images of legs that are uploaded to said central computer server from said plurality of end-user devices that comprise said first end-user device having said first level of bias and said second end-user device having said second level of bias;
wherein central updating of the DNN model, based on images of legs that are uploaded to said central computer server from the plurality of end-user devices that are located at a plurality of different locations and are operated by a plurality of different operators, causes the DNN model and detection of LLD to be more resilient to bias.

13. The computerized system of claim 1,
wherein at least one of the end-user devices is configured to capture a video clip that depicts legs of a person, and is further configured to select only a single particular video frame from said video clip;
wherein only said single particular video frame, and not other video frames of said video clip, is used for local in-device LLD detection;
wherein only said single particular video frame, and not other video frames of said video clip, is uploaded from said end-user device to a master LLD database of said central computer server;
wherein said single particular video frame is selected, locally within said end-user device, not based on its being a video frame having highest visible qualities to a human observer, but rather, based on being a video frame having the highest values of parameters that indicate image suitability for classification by a DNN-based classifier that classifies images based on manifestation or non-manifestation of LLD.

14. The computerized system of claim 1,
wherein at least some of the digital non-radiological non-X-Ray photographs, that are used for training the DNN model, include Body Landmarks indicators that are placed on particular body-parts or body-locations of humans that are depicted in said photographs;
wherein the DNN model is trained on a training set of images that include digital non-radiological non-X-Ray photographs that show include Body Landmarks indicators.

15. The computerized system of claim 1,
wherein at least some of the digital non-radiological non-X-Ray photographs, that are used for training the DNN model, include Augmented Reality Markers that are placed on particular body-parts or body-locations of humans that are depicted in said photographs;
wherein the DNN model is trained on a training set of images that include digital non-radiological non-X-Ray photographs that show include Augmented Reality Markers.

16. The computerized system of claim 1,
wherein said digital non-radiological non-X-Ray photographs, that are used for training the DNN model and/or for LLD classification, are stored in a blockchain that prevents content tampering.

17. The computerized system of claim 1,
wherein said digital non-radiological non-X-Ray photographs, that are used for training the DNN model and/or for LLD classification, are stored in a blockchain that prevents content tampering.

18. The computerized system of claim 1,
wherein the central computer server further comprises:
(A) an Over-Fitting Prevention Unit, that is configured to prevent or reduced over-fitting of the DNN model to digital non-radiological non-X-Ray photographs, (A1) by performing one or more randomly-selected transformations to existing images of pairs of legs, and creating a set of image variants which is used for increasing a diversity and a total number of training examples of pairs-of-legs, and also (A2) by removing one or more randomly-selected images from said existing images of pairs of legs during a training gradient or a training iteration;
(B) an Under-Fitting Prevention Unit, that is configured to prevent or reduce under-fitting of the DNN model relative to digital non-radiological non-X-Ray photographs, by performing at least one of: (B1) adding a hidden layer to the DNN model, (B2) modifying regularization parameters to the DNN model.

19. The computerized system of claim 1,
wherein the central computer server is configured to perform a process comprising:
determining whether a particular subject has a Leg Length Discrepancy (LLD), by performing:
(a1) receiving a training set of images of legs of patients;
(a2) receiving a validation set of images of legs of patients;
(b) operating on the training set of images by:
(b1) performing image normalization and image resizing on said images of legs of patients;
(b2) modifying the images of the training set, by applying one or more image transformation operations selected from the group consisting of: image rotation, image flip, skewing, zoom modification, isotropic scaling, shear transformation;
(b3) performing a binary-type classification of said images of legs of patients, into exactly one of: (i) a first class of images that includes only images that are determined to not be associated with LLD, or (ii) a second class of images that includes both images that are determined to be associated with LLD and images that are determined to possibly be associated with LLD;
(b4) passing the images of the training set of images via convolutions and extracting a first set of unique features from said images of the training set; and operating a Convolutional Neural Network (CNN) unit which applies convolution, kernel initialization, pooling, activation, padding, batch normalization, and stride to the images, to detect one or more particular image-features that are determined to be predictive for LLD detection;
(b5) perform pooling and image traversal, through a particular path of convolutions that was passed in step (b4), and concurrently extracting a next set of unique features from said images of the training set by using computerized-vision object detection and computerized-vision pattern recognition;
(b6) stacking multiple sets of convolutions that were passed in step (b4), and also stacking multiple pooling layers that were pooled in step (b5), to generate reduced-size images;
(b7) feeding the reduced-size images into one or more dense layers of said CNN unit;
(b8) applying a SoftMax classifier to reduce binary loss, and further applying a sigmoid classifier;
(b9) adjusting a learning rate of said CNN unit for convergence into a solution;
(b10) generating by said CNN unit a single-neuron output with a sigmoid activation, which indicates a binary-type output with regard to a particular image; wherein the binary-type output is either (i) the particular image is not associated with LLD, or (ii) the particular image is associated or is possibly associated with LLD;
(c) operating on the validation set of images by:
performing steps (b1) through (b10) on the validation set of images to verify an accuracy of classifications performed by said CNN unit;
(d) performing a transfer learning process at said central server, on a dynamically-updated dataset of images of legs of patients; periodically generating at said central server an upgraded DNN model; and periodically sending the upgraded DNN model to the plurality of imaging devices.

20. A computerized method,
which is implemented by utilizing at least: one or more processors that are configured to execute code, and that are operably associated with one or more memory units that are configured to execute code,
the computerized method comprising:
detecting Leg Length Discrepancy (LLD) of humans, by applying a Machine Learning algorithm with a Deep Neural Network (DNN) model that classifies digital non-radiological non-X-Ray photographs of legs;
wherein the computerized method comprises:
(a) providing a plurality of distributed end-user devices,
wherein each end-user device is an electronic device selected from the group consisting of: a smartphone, a tablet, an electronic device comprising a processor and an imager;
wherein each end-user device is configured to acquire digital non-radiological non-X-Ray photographs of legs of persons;
wherein each end-user device is configured to perform: (i) a learn-and-predict process, (ii) a Deep Neural Network (DNN) model upgrade process, and (iii) a database transfer process;
wherein each end-user device locally-stores therein, and locally-runs therein, a local version of a DNN model that is periodically updated by a central computer server;
(b) providing said central computer server, that is configured to communicate separately, over Internet-based communication links, with each one of the plurality of distributed end-user devices;
wherein the central computer server comprises a DNN Engine,
wherein the computerized method comprises operating said DNN Engine
(b1) to receive an initial training set of digital non-radiological non-X-Ray photographs of legs of persons,
(b2) to generate from said initial training set an initial DNN model, that is capable of classifying a particular new digital non-radiological non-X-Ray photograph either as manifesting LLD or as non-manifesting LLD,
(b3) to receive, from time to time, from a particular end-user device out of said plurality of end-user devices, a copy of additional digital non-radiological non-X-Ray photographs that were captured by said particular end-user device and that were already classified as manifesting LLD or non-manifesting LLD based on a current version of the DNN model that is installed in said particular end-user device,
(b4) to add said additional digital non-radiological non-X-Ray photographs to a master database utilized by said central computer server,
(b5) to update said initial DNN model based on cumulative DNN learning derived from said additional digital non-radiological non-X-Ray photographs;
wherein the computerized method further comprises:
at said DNN Engine, periodically upgrading the DNN model, and periodically distributing an upgraded DNN model to each one of said end-user devices;
wherein at least one of: (I) an end-user device out of the plurality of end-user devices, (II) said central computer server, is configured to utilize said upgraded DNN model to generate a determination for LLD diagnosis, indicating whether or not a particular subject has a Leg Length Discrepancy (LLD), by feeding a digital non-radiological non-X-Ray photograph of legs of said particular subject into said upgraded DNN model, based on output from a sigmoid-activated single-neuron of said DNN model.
